(19) United States
(12) Patent Application Publication
(10) Pub. No.: US 2024/0010683 A1
(43) Pub. Date: Jan. 11, 2024

(54) Peptide Inhibitors of Human Mitochondrial Fission Protein 1 and Methods of Use

(71) Applicant: The Medical College of Wisconsin, Inc., Milwaukee, WI (US)

(72) Inventors: John Michael Egner, Westmont, IL (US); R. Blake Hill, Milwaukee, WI (US); Michael E. Widlansky, Fox Point, WI (US); Kelsey Meacham, Brookfield, WI (US)

(21) Appl. No.: 18/252,073

(22) PCT Filed: Nov. 8, 2021
(86) PCT No.: PCT/US2021/058437

§ 371 Date: May 8, 2023

Related U.S. Application Data
(60) Provisional application No. 63/110,457, filed on Nov. 6, 2020.

(51) Int. Cl. C07K 7/08; C12N 15/63; A61P 9/08

(52) U.S. Cl. CPC C07K 7/08; C12N 15/63; A61P 9/08

(57) ABSTRACT

The present disclosure provides inhibitory peptides of mitochondrial fission protein 1 (Fis1), polynucleotides and vectors encoding the peptides, and methods of using the peptides to treat diseases, including arterial diseases and vascular dysfunction associated with type 2 diabetes.

### CROSS-REFERENCE TO RELATED APPLICATIONS

This application claims priority to U.S. Provisional Application No. 63/110,457 filed on Nov. 6, 2020, the contents of which are incorporated by reference in its entirety.

### STATEMENT REGARDING FEDERALLY SPONSORED RESEARCH

This invention was made with government support under R01 HL128240 and R01-GM067180 awarded by the National Institutes of Health. The government has certain rights in the invention.

### SEQUENCE LISTING

A Sequence Listing accompanies this application and is submitted as an ASCII text file of the sequence listing named “650053_00834_ST25.txt” which is 11.4 kbytes in size and was created on Nov. 5, 2021. The sequence listing is electronically submitted via EFS-Web with the application and is incorporated herein by reference in its entirety.

### BACKGROUND OF THE INVENTION

The field of the invention is related to fission protein 1 peptides, fusion peptides and methods for treating diseases, including vascular diseases and type 2 diabetes.

Impaired vascular endothelial function predates the development of macrovascular and microvascular disease in patients with type 2 diabetes (T2DM). Emerging data implicate abnormal mitochondrial form and function in the development of vascular endothelial dysfunction in humans with T2DM.^{1,2} Mitochondria in the endothelium of individuals with T2DM produce excessive superoxide. The excess superoxide is driven, in part, by greater polarization of the mitochondrial inner membrane.² Excessive mitochondrial reactive oxygen species (mtROS) production in the endothelium activates critical epigenetic changes and cell signaling pathways that lead to endothelial inflammation and vascular dysfunction.³ We have previously shown that mitochondrial-targeted antioxidants or pharmacological agents dosed to partially depolarize the mitochondrial inner membrane reverse impaired endothelium-dependent vasodilation in resistance arterioles from humans with T2DM.^{2,4}

Unfortunately, the phase 3 clinical trials of anti-oxidant therapeutic approaches to prevent and treat vascular diseases have failed to verify positive effects seen in smaller physiological and/or non-randomized studies,^{5,6} and current pharmacological agents that target the mitochondrial inner membrane have toxicity profiles that preclude their clinical use.⁷

Therefore there is a need for additional therapeutic approaches for targeting a treating vascular dysfunction, including in patients with type 2 diabetes.

### SUMMARY OF THE INVENTION

The present disclosure provides inhibitory peptides of mitochondrial fission protein 1 (Fis1) activity and methods of use.

In one aspect, the disclosure provides an inhibitory peptide of mitochondrial fission protein 1 (Fis1) activity comprising (a) an amino acid sequence of SEQ ID NO:38 (XLPYPZ) or a sequence having at least 80% sequence identity to SEQ ID NO:38, wherein X and Z can be a peptide from 0-30 amino acids, optionally from 1-20 amino acids. In some aspects, the inhibitory peptide comprisesan amino acid sequence selected from SEQ ID NO:33-37 or a sequence laving at least 80% sequence identity to SEQ ID NO:33-37, wherein the peptide is from about 5-50 amino acids in length, optionally 5-30 amino acids in length.

In another aspect, the disclosure provides a Fis1 inhibitory peptide comprising (a) an amino acid sequence of SEQ ID NO:1 (SHKHDPLPYPHFLL) or a sequence having at least 90% sequence identity to SEQ ID NO:1. In another aspect, the disclosure provides a Fis1 inhibitory peptide comprising (a) an amino acid sequence of SEQ ID NO:1 (SHKHDPLPYPHFLL) or a sequence having at least 90% sequence identity to SEQ ID NO:1 linked to (b) a carrier or an amino acid sequence encoding a carrier peptide, a tag peptide, or cell binding peptide.

In another aspect, the disclosure provides an inhibitor peptide of mitochondrial fission protein 1 (Fis1) activity comprising (a) an amino acid sequence of any one of SEQ ID NO:1, 16-21, 26 or 29 or a sequence having at least 80% sequence similarity, preferably at least 90% similarity to SEQ ID NO:1, 16-21, 26 or 29. In some aspects, the inhibitory peptide comprising (a) is linked to (b) a carrier or an amino acid sequence encoding a carrier peptide, a tag peptide, or cell binding peptide. In some aspects, the inhibitory peptide is linked or attached to a carrier peptide which is a cell penetrating peptide sequence, optionally TAT (SEQ ID NO:2) or a sequence having at least 80% sequence similarity, preferably at least 90% sequence identity to SEQ ID NO:2. In some aspects, both (a) and (b) are peptides and are linked by a linker amino acid sequence. In some aspects, the linker sequence is SEQ ID NO:4, 11, 12, 13, 14, or 15.

In another aspect, the disclosure provides a Fis1 inhibitory peptide comprising an amino acid sequence of SEQ ID NO:1 (SHKHDPLPYPHFLL) or a sequence having at least 80% preferably at least 90% sequence identity to SEQ ID NO:1. In another aspect, the Fis1 inhibitory peptide comprises SEQ ID NO:3 (YGRKKRRQRRRGSGSGSSHKHDPLPYPHFLL) or a peptide having at least 90% sequence identity to SEQ ID NO:3.

In another aspect, the inhibitory peptide comprises SEQ ID NO:3, SEQ ID NO:31, SEQ ID NO:32 or a peptide having at least 80% sequence similarity, preferably about 90% sequence identity to SEQ ID NO:3, SEQ ID NO:31 or SEQ ID NO:32.

In a further aspect, the disclosure provides a method of treating vascular complications associated with type 2 diabetes, the method comprises administering an effective amount of the Fis1 inhibitory peptide described herein to treat the vascular complications.

In another aspect, the disclosure provides a method of reversing impaired vasodilation in a subject in need thereof, the method comprising administering an effective amount of the Fis1 inhibitory peptide described herein to restore vasodilation in the subject. In an aspect, the subject has type 2 diabetes.

In another aspect, the disclosure provides a method of increasing NO bioavailability in human microvascular endothelial cells, the method comprising administering an effective amount of the Fis1 inhibitory peptide described herein to increase NO bioavailability in human endothelial cells.

### BRIEF DESCRIPTION OF THE DRAWINGS

FIG. 1: Vasodilation improved with Fis1 transfection compared to control (siRNA) with increased response to acetylcholine under both high and low glucose conditions. A. Impact of molecular inhibition of Fis1 expression on endothelial vasodilation under high glucose conditions. The impaired vasodilation caused by high glucose conditions was improved under Fis1 knockdown conditions (P=0.0002 overall, *P≤0.0002 at indicated Ach doses for control siRNA vs Fis1 siRNA, N=6 subjects). L-NAME could abolish this improvement of vasodilation under Fis1 knockdown conditions (P<0.0001 for Fis1 siRNA vs siFis1+L-NAME and P=0.002 for control siRNA vs control siRNA+L-NAME). B. Impact of molecular knockdown of Fis1 expression on endothelial vasodilation under low glucose conditions. The impaired vasodilation caused by low glucose conditions was improved under Fis1 knockdown conditions (P=0.0008 overall, *P≤0.0003 at indicated Ach doses for control siRNA vs Fis1 siRNA, N=6 subjects). L-NAME could abolish this improvement of vasodilation under Fis1 knockdown conditions (P=0.0002 for Fis1 siRNA vs Fis1 siRNA+L-NAME). Ach-Acetylcholine.

FIG. 2: The bioavailability of NO increased in human arterioles with reduction of Fis1 levels under high and low glucose conditions. A. Low glucose (LG): (n=8, overall P=0.01; P=0.03 for control siRNA vs. Fis1 siRNA; P=0.003 for control siRNA+L-NAME vs Fis1 siRNA; P=0.03 for Fis1 siRNA vs Fis1 siRNA+L-NAME. Box represents 25 and 75 percentiles. Horizontal line represents the median. B. High Glucose: (n=9, overall P=0.04; P=0.01 for scrambled siRNA vs Fis1 siRNA; P=0.03 for Fis1 siRNA vs control siRNA+L-NAME and P=0.047 for Fis1 siRNA vs Fis1 siRNA+L-NAME).

FIG. 3: Suppression of Fis1 expression in DM arterioles reversed impaired endothelium-dependent vasodilation (n=6, P=0.002 overall). L-NAME blocked this effect (P<0.0004 for Fis1 siRNA vs Fis1 siRNA+L-NAME). * P<0.0005 for vs. all other exposures at the indicated Ach doses. Ach-acetylcholine

FIG. 4: Molecular inhibition of Fis1 under high glucose (33 mM) and low glucose (2.5 mM) conditions improved steady-state junction stability between endothelial cells in the monolayer. Comparison of electric cell-substrate impedance sensing measurements (ECIS) in human microvascular endothelial cells (HMEC-1) transfected with Fis1 siRNA and control siRNA between high and normal Glucose conditions (A) and normal and low glucose conditions (B). Box represents 25th to 75th percentiles. Horizontal line represents the median. SANOVA followed by Tukey's multiple comparison test, n=4 for each treatment (P<0.001 overall for both high and low glucose studies. *−P<0.05, **P<0.01, ***P<0.001, ****P<0.0001. HG-High glucose, LG-Low Glucose, NG-Normal Glucose

FIG. 5: Molecular inhibition of Fis1 under high glucose (33 mM) and low glucose (2.5 mM) conditions did not alter mitochondrial bioenergetics compared to normal Glucose (5 mM) conditions. Measures of extracellular acidification rate (ECAR, n=5) (A) and oxygen consumption rate (OCR, n=5) (B) in human microvascular endothelial cells (HMEC-1) transfected with Fis1 siRNA and control siRNA pre-incubated with high glucose (6 hours), normal glucose (5 mM for 2 hours), and low glucose (2 hours). Wild type (WT) cells represent HMEC-1 cells not transfected and grown under normal glucose conditions. ECAR and OCR were measured under basal conditions followed by the sequential addition of oligomycin (2.5 μM), FCCP (1 μM), as well as rotenone (1 μM) and antimycin A (1 μM). No statistically significant differences were present between the treatments.

FIG. 6: Novel peptide pep213 binding to Fis1. (A)1H, 15N HSQC spectral overlay of a 50 μM 15N-Fis1 sample titrated with increasing amounts (0-2000 μM) of unlabeled p8470. (B) Affinity determination for pep213 binding to Fis1 from NMR data in (A) by applying TREND analysis to all spectra in the titration series and fitting the normalized principal component 1 values (PC1) to a ligand depletion model to determine an apparent KD=7±2 μM. (C) Affinity determination by intrinsic tryptophan fluorescence. Fis1 containing a single tryptophan was titrated with increasing amounts (0-1000 μM) of pep213 that contains no tryptophan. The average emission wavelength was determined and fit to determine an apparent KD=3.3±0.1 μM. Residuals to the fits in panels B and C are shown in the top of each panel.

FIG. 7: Pep213-tat reverses impaired endothelium dependent vasodilation in healthy human vessels exposed to high glucose and in vessels from humans with T2DM. (A) High glucose induced impairment of endothelium-dependent vasodilation to acetylcholine was reversed by treatment with 1-10 μM of pep213 (n=5, P<0.0001) attached to a tat sequence to facilitate cellular entry. L-NAME reversed all improvements with pep213-tat treatment (P<0.0001 for pep 213 vs pep-213+L-NAME). *P<0.0001 at the indicated Ach concentration for pep213 vs. all other exposures). B) The impaired endothelium-dependent vasodilation to Ach in vessels from T2DM subjects was reversed by pep213 (P<0.001 overall, *P<0.05 at the indicated Ach concentrations for pep213 vs. all other exposures). Ach-acetycholine. T2DM-type 2 diabetes

FIG. 8: pep213-tat reverses impaired endothelium dependent vasodilation in healthy human vessels exposed to high glucose and in vessels from humans with T2DM compared to scrambled peptide control. (A) Acetylcholine induced endothelium-dependent vasodilation pre-exposed to six hours of high glucose (33 mM) was reversed with 1 μM pep213-tat compared to a scrambled peptide containing the same amino acids as pep213 attached a tat sequence. (n=5, P<0.001 overall, *P<0.05 at indicated Ach concentrations). (B) Similar results were seen with vessels from T2DM subjects (n=4, P<0.001 overall, *P<0.05 at the indicated doses).

FIG. 9: Fis1 knockdown efficiency in human arterioles with siRNA treatment. Fis1 levels were significantly reduced (n=4, p<0.05) in human arterioles transfected with siRNA Fis1 compared to arterioles transfected with scrambled control siRNA.

FIG. 10: Transfection with Drp1 siRNA protected against high glucose-induced impairment in endothelium-dependent vasodilation. (P<0.0001 overall, *P≤0.0001 at indicated Ach doses for control siRNA vs Drp1 siRNA, n=6). L-NAME abolished this improvement of vasodilation under Drp1 knockdown conditions (P<0.0001 for Drp1 siRNA vs.Drp1 siRNA+L-NAME).

FIG. 11. Knockdown of Drp1 expression with Drp1 siRNA protected against reductions in nitric oxide (NO) bioavailability in arterioles from healthy humans human arterioles exposed to high or low glucose conditions. (A) High glucose (HG, 33 mM, six-hour exposure): n=9, P=0.02 overall; P<0.05 for Drp1 siRNA vs. all other exposures. (B) Low glucose (LG, 2.5 mM, two-hour exposure): n=5; overall P=0.003, P=0.03 or less for Drp1 siRNA vs. all other exposures.

FIG. 12. Suppression of Drp1 expression in arterioles from humans with T2DM using Drp1 siRNA tended to reverse impaired endothelium-dependent vasodilation (n=4, P=0.076).

FIG. 13. Fis1 knockdown efficiency in HMEC-1 cells with siRNA treatment. Fis1 levels were significantly reduced (n=3, overall P<0.0001) in HMEC-1 cells transfected with Fis1 siRNA Fis1 compared to cells transfected with scrambled control siRNA (P=0.0002) and non-transfected HMEC-1 cells (P=0.0001).

FIG. 14. Phosphorylation of eNOS at Ser1177 and NO production increased in HMEC-1 cells upon activation with Ca+2 ionophore A23187 (A) representative western blot of p-eNOS-Ser1177) and β-actin with and without addition of A23187.(B) Quantitative measure of p-eNOS-(Ser1177) treated with and without A23187 (P=0.03, n=6). (C) NO production as measured using DAF2-DA (5 uM) increased upon addition of A23187 (n=4, P=0.02).

FIG. 15. Fis1 knockdown improved NO production in immortalized cultured human microvascular endothelial cells (HMEC-1) transfected with Fis1 siRNA. n=7, P<0.0001 overall; siFis1 basal vs siFis1 stimulated-P<0.0001; siRNA basal vs siFis1 stimulated-P=0.0003; siFis1 stimulated vs siRNA stimulated-P=0.02). Cells exposed to L-NAME were incubated with L-NAME for 2 hours followed by a 15-minute incubation with DAF2-DA (5 uM) prior to measurement of fluorescence intensity.

FIG. 16. Molecular inhibition of Fis1 under high glucose (33 mM) and low glucose (2.5 mM) conditions did not alter the expression of other mitochondrial proteins. The expression of selected mitochondrial proteins was measured from immortalized HMEC-1 cells transfected with siRNA Fis1 or scrambled siRNA and pre-incubated with different glucose conditions: high glucose (HG, 33 mM for six hours), normal glucose (NG, 5 mM for 2 hours) and low glucose (LG, 2.5 mM for 2 hours). The expression of each protein was normalized to total protein across the samples. (n=4-10 for individual proteins). While there are differences in expression of some mitochondrial proteins seen when comparing different glucose concentration exposures, knockdown of Fis1 expression with siRNA did not affect expression of any mitochondrial protein except for Fis1. Statistically significant differences are indicated by * for p<0.05, ** for p<0.01, *** for p<0.001, and **** for p<0.0001.

FIG. 17. The bioavailability of nitric oxide (NO) increased in human microvascular endothelial cells (HMVEC) treated with novel peptide pep213. HMVEC cells were treated with 1 μM pep213-tat for one hour at 37° C. and NO was measured with diaminofluorescein-2 diacetate (DAF2-DA) staining. N=3,* P=0.04.

FIG. 18. Impaired endothelium-dependent vasodilation in arterioles overexpressing Fis1 is eNOS-dependent. Overexpression of Fis1 in resistance arterioles from healthy human (transfected with a plasmid for endothelial-specific over-expression of human Fis1 with a 48 hour incubation period) results in impaired endothelium-dependent vasodilation in an eNOS-dependent manner (as determined by the loss in acetylcholine induced endothelium-dependent vasodilation with the use of eNOS inhibitor L-NAME). N=5, P<0.001 overall. *P<0.05 at the indicated doses of acetylcholine. Ach-acetylcholine

FIG. 19. Pep213 can reverse impaired endothelium-dependnet vasodilation in resistance arterioles. One hour of exposure to pep213 attached to a tat sequence to improve cell penetration (1 μM pep213-tat) reverses impaired endothelium-dependent vasodilation in resistance arterioles from healthy humans over-expressing Fis1 in the endothelium (overexpression of human Fis1 achieved using lentiviral vector to transfect the vessel with a plasmid for endothelial-specific over-expression of human Fis1). 1 μM of a scrambled peptide using the same amino acids as pep213 in a random order has no effect on endothelium dependent vasodilation to acetylcholine. Pep213-tat induce improves endothelium-dependent vasodilation in an eNOS-dependent manner (as determined by the loss in acetylcholine induced endothelium-dependent vasodilation with the use of eNOS inhibitor L-NAME). N=5, P<0.001 overall. *P<0.05 at the indicated doses of acetylcholine. Ach-acetylcholine.

FIG. 20. Crystal structure of pep213 and Fis1 and peptide mapping of Pep213. (A) The co-complex structure clearly shows pep213 engaging with Fis1 via a variety of bonding interactions including salt-bridge formation, hydrogen bonding, and Van der Waals interactions. (B) Microscale thermophoresis was used to determine critical pep213 residues for the Fis1-pep213 interaction. Each residue in pep213 was sequentially replaced with an alanine, for a total of 14 peptides. Binding affinity values were used to determine the AG° of the reaction (AG°=-RTlnK) which was then used to calculate a ΔΔG° value (ΔG°ₚₑₚ₂₁₃-ΔG°ᵥₐᵣᵢₐₙₜ). A subset of residues on each terminus of the peptide do not contribute significantly to binding, as indicated by a lower ΔΔG°⁰ value (B).

### DETAILED DESCRIPTION OF THE INVENTION

The present invention provides peptides, nucleic acid sequences and vectors encoding the peptides, compositions containing the peptides or vectors, and methods of using them to treat diseases associated with endothelial or vascular dysfunction, including type 2 diabetes mellitus (T2DM) and vascular diseases.

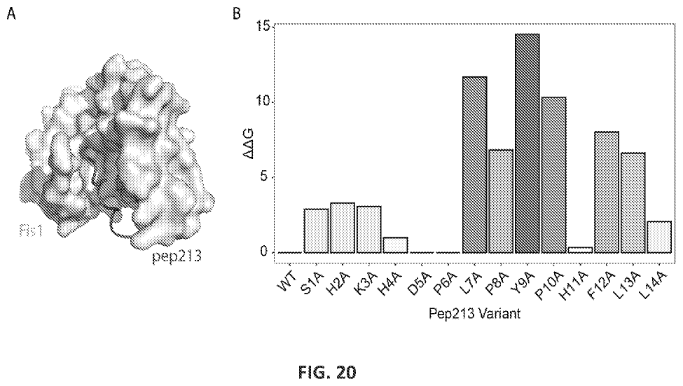

Excessive mitochondrial fission has been implicated in a variety of diseases including endothelial dysfunction in diabetes mellitus. Mitochondrial fission is the fragmentation of mitochondria into smaller mitochondrial units, and genetic or pharmacological inhibition of fission improves vasodilation ex vivo. This inhibition includes RNAi-mediated genetic silencing of the gene encoding mitochondrial fission protein I (Fis 1). These data suggest that inhibition of Fis 1 may improve pathological conditions where vasodilation is impaired. To identify inhibitors of Fis 1, we have developed a high-affinity 14-residue peptide, pep213 (SEQ ID NO:1), that binds to recombinant Fis1 with micromolar to sub-micromolar affinity. Application of a cell permeable version of pep213 to human endothelial vessels recovers vasodilation suggesting that the peptide inhibits Fis1 activity in vivo and may have therapeutic value. Pep213 is a novel peptide that derives from peptides from a phage display screen to bind to a severely truncated form of Fis1 lacking the first 32 residues. Further, we did co-crystalization and mutatgenesis analysis to hone in on the core amino acids within pep213 necessary for binding to Fis1 (see FIG. 20).

Targeting proteins and peptides involved in mitochondrial fission represent a promising alternative approach based on prior work demonstrating (1) increased expression of mitochondrial fission 1 protein (Fis1) in endothelial cells obtained from humans with type 2 diabetes; (2) molecular knockdown of Fis1 or dynamin-related protein 1 (Drp1, which may bind Fis1 to induce mitochondrial fission) expression blocks high-glucose induced increases in mitochondrial superoxide production and impairment of phosphorylation of endothelium-derived nitric oxide synthase (eNOS) at its Serl177 activation site; and (3) pharmacological and molecular knockdown of Drp1 reverse low-glucose induced endothelial dysfunction in human resistance arterioles.^{1,8} Fis1 as a pharmacological target is of particular interest given its role in mitochondrial dynamics appears to be most highly active in the setting of pathological stimuli like hypoxemia and hyperglycemia.⁹⁻¹²

The present disclosure, as described in the Examples, tested whether knockdown of Fis1 expression reversed impaired endothelium-dependent vasodilation and nitric oxide (NO) production in resistance vessels from patients with type 2 diabetes and in vessels from healthy individuals exposed acutely to high and low glucose concentrations. Additionally, the effect of Fis1 knockdown on endothelial cell barrier function, oxygen consumption, and glycolysis under high or low glucose conditions was determined. The inventors then designed and tested a novel peptide engineered to bind to Fis1 and block Fis-1 mediated fission favorably impacted endothelium-dependent vasodilation of small resistance arteries from humans with T2DM and from healthy human vessels exposed to high glucose concentrations. Pharmacological targeting of Fis1 provides a useful therapeutic avenue for the challenge of vascular disease in T2DM.

### Peptides and Compositions

The present invention provides a novel peptide that binds to recombinant Fis1, preferably with micromolar to sub-micromolar affinity.

In a first embodiment, the disclosure provides an inhibitory peptide of mitochondrial fission protein 1 (Fis1) activity comprising (a) an amino acid sequence of SEQ ID NO:38 (XLPYPZ) or a sequence having at least 80% sequence identity to SEQ ID NO:38, wherein X and Z can be a peptide from 0-30 amino acids, optionally from 1-20 amino acids, optionally from 1-10 amino acids. In another embodiment, the amino acid sequence of SEQ ID NO:38 or a sequence having at least 90% sequence identity. In another aspect, the inhibitory peptide of claim 1 comprising (a) an amino acid sequence selected from SEQ ID NO:33-37 or a sequence having at least 80% sequence identity or at least 90% sequence identity to SEQ ID NO:33-37, wherein the peptide is from about 5-50 amino acids in length, optionally 5-30 amino acids in length. Preferably amino acid lengths may be about 10 to 20 amino acids, or about 12-16 amino acids in length. Other suitable lengths are contemplated. Suitably, the (a) is linked to (b) a carrier peptide or tag, described more below.

In a further embodiment, a 14-mer peptide, pep213 (SEQ ID NO:1, 16-21, 26 or 29, preferably in one embodiment SEQ ID NO:1) or a sequence having at least 80% sequence identity, optionally at least 90% sequence identity, when made into a cell permeable fusion peptide (e.g., pep213-TAT, SEQ ID NO:3 or SEQ ID NO:31 or 32 or sequences having at least 80% or at least 90% sequence identity to SEQ ID NO:3, 31, or 32) are capable of inhibiting Fis1 activity in vivo. This inhibitory peptide is also able to recover vasodilation in endothelial cells and vessels in which vasodilation is impaired. The ability of the peptides to reverse impaired endothelium-dependent vasodilation further allows for the peptide to be used to treat vascular diseases, including vascular dysfunction associated with type 2 diabetes. Further, co-crystallization and peptide mutational analysis as seen in FIGS. 19 and 20 demonstrates the important amino acids within the 14-mer for Fis1 binding. Thus, in some aspects, modified pep213 peptides are contemplated (e.g., SEQ ID NO:16-29, preferably SEQ ID NO:16-21, 26 or 29 or SEQ ID NO:30 having one or more of the amino acids X replaced by any amino acid, preferably an alanine or glycine as the X).

In one embodiment, the inhibitory peptide of mitochondrial fission protein 1 (Fis1) activity comprises, consists of a inhibitory peptide described herein linked to a carrier peptide, a tag peptide, or a cell binding peptide. In one aspect, the inhibitory peptide of mitochondrial fission protein 1 (Fis1) activity comprising (a) an amino acid sequence of SEQ ID NO:38 (XPLPYPZ) or a sequence having at least 80% sequence identity to SEQ ID NO:38, wherein X and Z can be a peptide from 0-30 amino acids, optionally from 1-20 amino acids, optionally from 1-10 amino acids (the amino acid peptide may comprise any suitable amino acids). In another embodiment, the amino acid sequence of SEQ ID NO:38 or a sequence having at least 90% sequence identity. In another aspect, the inhibitory peptide of claim 1 comprising (a) an amino acid sequence selected from SEQ ID NO:33-37 or a sequence laving at least 80% sequence identity or at least 90% sequence identity to SEQ ID NO:33-37, wherein the peptide is from about 5-50 amino acids in length, optionally 5-30 amino acids in length. In another aspect, the inhibitor comprises or consists of (a) an amino acid sequence of SEQ ID NO:1 (SHKHDPLPYPHFLL) or is a sequence having at least 90% sequence identity to SEQ ID NO:1. In another embodiment, the inhibitory peptide of Fis1 comprises, consists of (a) an amino acid sequence of SEQ ID NO:1 or a sequence having at least 90% sequence identity to SEQ ID NO:1 linked to (b) a carrier or an amino acid sequence encoding a carrier peptide, a tag peptide, or cell binding peptide. The term “inhibitory peptide of Fis1” and “Fis1 inhibitory peptide” are used herein interchangeably and refer to a peptide that is capable of inhibiting the activity of Fis1 within a cell.

In one embodiment, the inhibitory peptide of mitochondrial fission protein 1 (Fis1) activity comprises, consists of or is (a) an amino acid sequence of SEQ ID NO:1, 16-21, 26 or 29 or is a sequence having at least 80% sequence identity or at least 90% sequence identity to SEQ ID NO:1, 16-21, 26 or 29. In another embodiment, the inhibitory peptide of Fis1 comprises, consists of (a) an amino acid sequence of SEQ ID NO:1, 16-21, 26 or 29 or a sequence having at least 80% sequence identity or at least 90% sequence identity to SEQ ID NO:1, 16-21, 26 or 29 linked to (b) a carrier or an amino acid sequence encoding a carrier peptide, a tag peptide, or cell binding peptide.

In another embodiment, based on mutant peptide analysis demonstrated in FIG. 20, the inhibitory peptide of mitochondrial fission protein 1 (Fis1) activity comprises, consists of or is (a) an amino acid sequence of SEQ ID NO:30, wherein one or more of the X is any amino acid (for example, an alanine or glycine) or the corresponding amino acids from SEQ ID NO:1. In another embodiment, the inhibitor peptide comprises or consists of SEQ ID NO:30 with two or more Xs being any amino acid, alternatively, 3 or more X are any amino acid, alternatively 4 or more X are any amino acid, alternative 5 or more X are any amino acid, alternatively 6 or more Xs are any amino acid, alternatively 7 or 8 X are any amino acid.

In another embodiment, the inhibitory peptide comprises, consists of (a) an amino acid sequence of SEQ ID NO:30 linked to (b) a carrier or an amino acid sequence encoding a carrier peptide, a tag peptide, or cell binding peptide, for example, SEQ ID NO:32.

The term “inhibitory peptide of Fis1” and “Fis1 inhibitory peptide” are used herein interchangeably and refer to a peptide that is capable of inhibiting the activity of Fis1 within a cell.

The inhibitory peptide of Fis1 further comprises a carrier or a carrier peptide, tag peptide or cell binding peptide linked to the inhibitory peptide. Suitable carrier peptides, tag peptides, or cell binding peptides are known and understood in the art. In one embodiment, the carrier peptide is a cell penetrating peptide. Cell penetrating peptides (CPP) are peptides that are able to penetrate the plasma membrane and reach the inside of a cell. For example, a suitable carrier peptide includes, for example, TAT, having amino acid sequence of SEQ ID NO:2 or a sequence having at least 90% sequence identity to SEQ ID NO:2 and able to penetrate into a cell. Other suitable CPPs are known in the art and include, for example, Penetratin, R8, Transportan, Xentry (see, e.g., Patel, S. G., Sayers, E. J., He, L. et al. Cell-penetrating peptide sequence and modification dependent uptake and subcellular distribution of green florescent protein in different cell lines. Sci Rep 9, 6298 (2019). //doi.org/10.1038/s41598-019-42456-8, incorporated by reference.) Other suitable carriers are known in the art. Other carriers include, but are not limited to, for example, nanocarriers, such as, polymer conjugates, polymeric nanoparticles, lipid-based carriers, dendrimers, carbon nanotubes, and gold nanoparticles. Lipid-based carriers include both liposomes and micelles. The carriers can be linked either covalently or non-covalently. In some embodiments, the peptide may be conjugated to the carrier.

In some embodiments, the peptides described herein further comprise an exogenous tag or agent. The term “tag” or “agent” as used herein includes any useful moiety that allows for the purification, identification, detection, or therapeutic use of the peptides of the present invention. Any tag or agent that does not interfere with the functionality of the inhibitory peptides may be used with the present invention. Suitable tags are known in the art and include, but are not limited to, affinity or epitope tags (e.g., cMyc, HIS, FLAG, V5-tag, HA-tag, NE-tag, S-tag, Ty tag, etc) and florescence tags (e.g., RFP, GFP, etc). Epitope tags are commonly used as a “purification tag”, i.e. a tag that facilitates isolation of the polypeptide from other non-specific proteins and peptides.

In some embodiments, the carrier peptide or tag is a polypeptide and the inhibitory peptide and the tag are encoded in one nucleic acid sequence and translated concurrently. In some embodiments, the tag is cleavable and can be removed once the peptide is made and purified.

In some embodiments, the inhibitory peptide and carrier peptide or tag are linked via a linker sequence. Suitable peptide linkers can comprise a polypeptide of 3 to 10 amino acids, or 3 to 25 amino acids. In some embodiments, the peptide linker comprises a polypeptide having an amino acid sequence selected from serines and glycines, for example GSGSGS (SEQ ID NO:4). Other suitable linkers would be understood by one skilled in the art and include, for example, SGSG (SEQ ID NO:11), Gₙ wherein n is an integer from 1 to 10, (SGSG)ₙ wherein n is an integer from 1 to 10, (SEQ ID NO:11), GSGS (SEQ ID NO:12), SSSS (SEQ ID NO:13), GGGS (SEQ ID NO:14), GGC, GGS, (GGC)₈), (G₄S)₃, and GGAAY (SEQ ID NO:15). The peptide linker may be cleavable by a protease. In some embodiments, the peptide linker comprises a polypeptide having an amino acid sequence of SEQ ID NO:4. Other suitable linkers known in the art are contemplated for use herein.

In one embodiment, the Fis1 inhibitory peptide comprises, consists of or is SEQ ID NO:3 (YGRKKRRQRRRGSGSGSSHKHDPLPYPHFLL) or a peptide having at least 90% sequence identity to SEQ ID NO:3. As demonstrated in the examples, this inhibitory peptide is able to inhibit Fis1 activity in vivo.

In another embodiment, the Fis1 inhibitory peptide comprises, consists of or is SEQ ID NO:31 (YGRKKRRQRRRGSXSHKHDPLPYPHFLL) or a peptide having at least 80% sequent similarity or at least 90% sequence identity to SEQ ID NO:31, wherein X is a linker described herein. In another embodiment, the FIs1 inhibitor peptide comprises SEQ ID NO:32 or a sequence having at least 80% sequence similarity or at least 90% sequence identity to SEQ ID NO:32, wherein at least one X is an any amino acid (for example, alanine) and wherein Y is a linker as described herein. (See Table 2). In some embodiments, it is contemplated that SEQ ID NO:32 has X comprising two or more amino acids selected from any amino acid (e.g. alanine), alternatively 3 or more, alternatively 4 or more, alternatively 5 amino acids selected from any amino acid (e.g. alanine) within the sequence, alternatively 6 or more X are amino acids selected from any amino acid (e.g. alanine), alternatively 7 or 8 X are amino acids selected from any amino acid (e.g. alanine) within SEQ ID NO:32. Y is a linker as described herein, for example, SEQ ID NO:4, or 11-15. In other words, SEQ ID NO:32 is SEQ ID NO:2-linker-SEQ ID NO:30 and SEQ ID NO:31 is SEQ ID NO:2-linker-SEQ ID NO:1. Linker can be any linker described herein.

As used herein, the terms “proteins,” “peptides” and “polypeptides” are used interchangeably herein to designate a series of amino acid residues connected to the other by peptide bonds between the alpha-amino and carboxy groups of adjacent residues. “Protein” and “polypeptide” are often used in reference to relatively large polypeptides, whereas the term “peptide” is often used in reference to small polypeptides, but usage of these terms in the art overlaps. Proteins may include modified amino acids (e.g., phosphorylated, glycated, glycosylated, etc.) and amino acid analogs.

The inhibitory peptides may be linked directly, linked indirectly, or conjugated to the tag or carrier peptide. As used herein, the term “conjugate” refers to the joining of two entities by covalent bonds. The entities may be covalently bonded directly or through linking groups using standard synthetic coupling procedures. For example, two polypeptides may be linked together by simultaneous polypeptide expression, forming a fusion or chimeric protein. One or more amino acids may be inserted into the polypeptide to serve as a linking group (i.e., via incorporation of corresponding nucleic acid sequences into the vector). For example, in some embodiments, a polyserine and polyglycine linker is included between the inhibitory peptide sequence and a tag or carrier peptide. Other contemplated linking groups include polyethylene glycols or hydrocarbons terminally substituted with amino or carboxylic acid groups to allow for amide coupling with polypeptides having amino acids side chains with carboxylic acid or amino groups, respectively. Alternatively, the amino and carboxylic acid groups can be substituted with other binding partners, such as an azide and an alkyne groups, which undergo copper catalyzed formation of triazoles.

The present disclosure also provides polynucleotides encoding the inhibitory Fis1 peptides disclosed herein. The terms “polynucleotide,” “polynucleotide sequence,” “oligonucleotide,” “nucleic acid,” and “nucleic acid sequence” are used interchangeably herein to refer to nucleotide sequences or fragments thereof. These phrases may refer to DNA or RNA of genomic, natural, or synthetic origin, and include single-stranded or double-stranded molecules, as well as sense or antisense strands of such molecules. In one embodiment, the polynucleotide comprising heterologous promoter sequence and a polynucleotide sequence encoding the peptide of SEQ ID NO:1 (SHKHDPLPYPHFLL), SEQ ID NO:16-21, 26 or 29 or a sequence having at least 80% sequence identity or at least 90% sequence identity to SEQ ID NO:1, 16-21, 26 or 29. In another embodiment, the polynucleotide comprising heterologous promoter sequence and a polynucleotide sequence encoding the peptide of SEQ ID NO:1 linked to a carrier or tag peptide, for example, SEQ ID NO:3, 31 or 32 or a sequence having at least 80% sequence identity or at least 90% sequence identity to SEQ ID NO:3, 31 or 32.

In one embodiment, the polynucleotide comprising heterologous promoter sequence and a polynucleotide sequence encoding the peptide of SEQ ID NO:1 (SHKHDPLPYPHFLL) or a sequence having at least 80% sequence identity or at least 90% sequence identity to SEQ ID NO:1. In another embodiment, the polynucleotide comprising heterologous promoter sequence and a polynucleotide sequence encoding the peptide of SEQ ID NO:1 linked to a carrier or tag peptide, for example, SEQ ID NO:3 or a sequence having at least 80% sequence identity or at least 90% sequence identity to SEQ ID NO:3.

In terms of sequence identity as used above and herein, anywhere citing at least 80% sequence identity includes any sequence identity about 80%, for example at least 81%, 82%, 83%, 84%, 85%, 86%, 87%, 88%, 89%, 90%, 91%, 92%, 93%, 94%, 95%, 96%, 97%, 98%, 99%, and 100%. Similarly, at least 90% sequence identity includes sequence identity about 90% and above, for example, 91%, 92%, 93%, 94%, 95%, 96%, 97%, 98%, 99%, and 100% sequence identity to the SEQ ID NO.

In some embodiments, the polynucleotide is a vector. In some embodiments, the vector is capable of expressing the inhibitory peptide described herein, wherein the vector comprises a heterologous promoter operably connected to a polynucleotide sequence encoding the inhibitory Fis1 peptide described herein. The vector may further comprise heterologous backbone sequence. Suitable vectors for use with the present invention comprise a promoter operably connected to a polynucleotide sequence encoding the inhibitory peptides described herein. The vectors may also comprise appropriate control sequences that allow for translational regulation in a host cell. In some embodiments, the vectors further comprise the nucleic acid sequences for one or more carrier peptides or tags. In some embodiments, the vectors further comprise additional regulatory sequences, such as signal sequences.

As used herein, the term “vector” refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors” (or simply, “vectors”). The term vector encompasses “plasmids”, the most commonly used form of vector. Plasmids are circular double-stranded DNA loops into which additional DNA segments (e.g., those encoding inhibitory Fis1 peptides) may be ligated. However, other forms of expression vectors, such as viral vectors (e.g., replication defective retroviruses, adenoviruses and adena-associated viruses), may also be used with the present invention. Certain vectors are capable of autonomous replication in a host cell into which they are introduced (e.g., bacterial vectors having a bacterial origin of replication and episomal mammalian vectors). Other vectors may be integrated into the genome of a host cell upon introduction into the host cell, and are thereby replicated along with the host genome. In one embodiment, the vectors comprise viral vectors that use viral machinery to carry the peptide to be expressed in a host cell.

In some embodiments, the vectors of the present invention further comprise heterologous backbone sequence. As used herein, “heterologous nucleic acid sequence” refers to a non-human nucleic acid sequence, for example, a bacterial, viral, or other non-human nucleic acid sequence that is not naturally found in a human. Heterologous backbone sequences may be necessary for propagation of the vector and/or expression of the encoded peptide. Many commonly used expression vectors and plasmids contain non-human nucleic acid sequences, including, for example, CMV promoters.

Suitable promoters for the practice of the present invention include, without limitation, constitutive, inducible, temporally regulated, developmentally regulated, chemically regulated, physically regulated (e.g., light regulated or temperature-regulated), tissue-preferred, and tissue-specific promoters. Suitable promoters include “heterologous promoters”, a term that refers to any promoter that is not naturally associated with a polynucleotide to which it is operably connected. In mammalian cells, typical promoters include, without limitation, promoters for Rous sarcoma virus (RSV), human immunodeficiency virus (HIV-1), cytomegalovirus (CMV), SV40 virus, and the like as well as the translational elongation factor EF-1α promoter or ubiquitin promoter. Those of skill in the art are familiar with a wide variety of additional promoters for use in various cell types.

Protein and nucleic acid sequence identities are evaluated using the Basic Local Alignment Search Tool (“BLAST”) which is well known in the art (Karlin and Altschul, 1990, Proc. Natl. Acad. Sci. USA 87: 2267-2268; Altschul et al., 1997, Nucl. Acids Res. 25: 3389-3402). The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. Preferably, the statistical significance of a high-scoring segment pair is evaluated using the statistical significance formula (Karlin and Altschul, 1990), the disclosure of which is incorporated by reference in its entirety. The BLAST programs can be used with the default parameters or with modified parameters provided by the user.

“Percentage of sequence identity” or “percent similarity” is determined by comparing two optimally aligned sequences over a comparison window, wherein the portion of the polynucleotide or peptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” or “substantial similarity” of polynucleotide or peptide sequences means that a polynucleotide or peptide comprises a sequence that has at least 75% sequence identity. Alternatively, percent identity can be any integer from 75% to 100%. More preferred embodiments include at least: 75%, 80%, 85%, 86%, 87%, 88%, 89%, 90%, 91%, 92%, 93%, 94%, 95%, 96%, 97%, 98% or 99% compared to a reference sequence using the programs described herein; preferably BLAST using standard parameters, as described. These values can be appropriately adjusted to determine corresponding identity of proteins encoded by two nucleotide sequences by taking into account codon degeneracy, amino acid similarity, reading frame positioning and the like.

“Substantial identity” of amino acid sequences for purposes of this invention normally means polypeptide sequence identity of at least 75%. Preferred percent identity of polypeptides can be any integer from 75% to 100%. More preferred embodiments include at least 75%, 80%, 85%, 86%, 87%, 88%, 89%, 90%, 91%, 92%, 93%, 94%, 95%, 96%, 97%, 98%, 98.7%, or 99%.

The present disclosure also provides compositions comprising the inhibitory Fis1 peptides and a pharmaceutically acceptable carrier. The pharmaceutically acceptable carrier should be selected based on the selected route of administration and standard pharmaceutical practice. The composition may be formulated into dosage forms according to standard practices in the field of pharmaceutical preparations (See Alphonso Gennaro, ed., Remington's Pharmaceutical Sciences, 18th Ed., (1990) Mack Publishing Co., Easton, Pa). Suitable dosage forms may comprise, for example, solutions, parenteral solutions, or suspensions. In some embodiments, the composition comprises an isolated and purified Fis1 inhibitory peptide described herein. In other embodiments, the composition comprises an isolated and purified polypeptide or vector comprising the nucleic acid sequences encoding a Fis1 inhibitory peptide described herein.

In another aspect, the present disclosure provides host cells comprising the vectors described herein. Any host cell that allows for expression of the peptides encoded by the vectors may be used with the present invention. For example, common host cells include bacteria (e.g., E. coli, B. subtilis), yeast (e.g., S. cerevisiae) or eukaryotic cell lines. Advantageously, insect or mammalian cell lines may be used to provide human-like splicing of mRNA. Those of skill in the art are aware that many expression systems and cell lines may be used to express the peptides of the present invention, including many that are commercially available.

### Methods

The present disclosure provides methods of treating vascular dysfunction using the Fis1 inhibitory peptides described herein. The method comprises administering an effective amount of the Fis1 inhibitory peptide described herein to treat the vascular dysfunction. In some embodiments, the vascular dysfunction is associated with type 2 diabetes. In some embodiments, the disclosure provides a method of treating vascular complications associated with type 2 diabetes. The method comprises administering an effective amount of the Fis1 inhibitory peptide described herein to reduce one or more vascular complication associated with type 2 diabetes.

The present disclosure further provides a method of reversing impaired vasodilation in a subject in need thereof, the method comprising administering an effective amount of the Fis1 inhibitory peptide described herein to restore vasodilation function within the subject. In some embodiments, the subject has type 2 diabetes. Further, the subject can have high glucose-induced and type 2 diabetes-associated impairment of endothelium-dependent vasodilation in human resistance arteries. This impairment can be nitric oxide synthase-dependent manner.

In another embodiment, the disclosure provides a method of increasing NO bioavailability in human microvascular endothelial cells, the method comprising administering an effective amount of the Fis1 inhibitory peptide described herein to increase NO bioavailability in human endothelial cells. In some embodiments, the endothelial cells are in vivo in a subject having vascular dysfunction.

In another embodiment, the disclosure provides a method of protecting against diabetic-induced cellular damage to endothelial tissue, the method comprising administering an effective amount of the Fis1 inhibitory peptide described herein. Excessive mitochondrial fission has been implicated in endothelial tissue dysfunction in diabetes, and the reduction in Fis1 can be protective against diabetic-induced cellular damage.

In a further embodiment, the disclosure provides a method of treating ras-mediated cancers which have been shown to require mitochondrial fission. The method comprises administering an effective amount of the Fis1 inhibitory peptide described herein. Not to be bound by any theory, but the ability to blocking mitochondrial fission, can stops cancer progression suggesting that inhibitors of Fis1 may be active against cancer progression.

In another embodiment, the Fis1 inhibitory peptides may be used to treat neurodegenerative diseases. The method comprises administering an effective amount of the Fis1 inhibitory peptide described herein to treat the neurodegenerative disease.

The present Fis1 inhibitory peptide is believed to be a better target that Drp for a number of reasons. First, the target is Drp1, which is embryonic lethal and is the sole mechanoenzyme in fission and may not be a suitable target except in cancer. Fis1 knockout is also embryonic lethal but is thought to be induced in only stress conditions and therefore might be a better target than Drp1.

Excessive mitochondrial fission has been implicated in endothelial tissue dysfunction in diabetes (Shenouda, S. M., Widlansky, M. E., Chen, K., Xu, G., Holbrook, M., Tabit, C. E., . . . & Vita, J. A. (2011) and Kizhakekuttu . . . Widlansky et al (2012)] and acute pulmonary dysfunction. Targeting Fis1 would allow for a novel pharmacological approach of the Fis1-Drp1 axis to protect against vasculature damage in diabetic patients. As mentioned above, targeting the Fis1/Drp1 axis through a peptide inhibitor of Drp1 has had numerous claims in neurodegenerative diseases as well as cardiomyopathies.

In a further embodiment, the disclosure provides method of treating impaired endothelial function, the method comprising the method comprising administering an effective amount of the Fis1 inhibitory peptide described herein in order to treat the impaired endothelial function. In one embodiment, the impaired endothelial function comprises atherosclerosis. In another embodiment, the impaired endothelial function is associated with a disease selected from atherosclerosis, cerebrovascular arterial disease, coronary arterial disease, renovascular disease, and peripheral arterial disease.

The terms “effective amount” or “therapeutically effective amount” refer to an amount sufficient to effect beneficial or desirable biological and/or clinical results. For example, therapeutically effective amounts of the peptides of the instant invention may be combined with a pharmaceutically acceptable carrier to form a composition. The composition can be administered in any of the art-recognized modes. The doses, methods of administration, and suitable pharmaceutically acceptable carriers, diluents, and excipients for use with such methods can readily be determined by a skilled artisan, but will depend on the particular circumstances at hand.

Appropriate dosages may be determined, for example, by extrapolation from animal studies or in clinical trials taking into account body weight of the patient, absorption rate, half-life, disease severity and the like. The number of doses and course of treatment may be varied from individual to individual. In some embodiments for the prevention of the development or progression of an autoimmune disease, booster dosages may be required. Suitable booster schedules may be determined by a skilled artisan. For example, the peptides or vectors may be given every month, every other month, every 4 months, every 6 months, once a year, once every two years, and any range of time in between.

The composition is preferably in unit dosage form. In such form, the preparation is divided into unit doses containing appropriate quantities of the active component. The unit dosage form can be a packaged preparation, the package containing discrete quantities of preparation, such as packeted tablets, capsules, and powders in vials or ampoules. In addition, the unit dosage form can be a capsule, tablet, cachet, or lozenge itself, or it can be the appropriate number of any of these in packaged form.

As used herein, “subject” or “patient” refers to both mammals and non-mammals. “Mammals” include any member of the class Mammalia, such as humans, non-human primates (e.g., chimpanzees, other apes and monkey species), farm animals (e.g., cattle, horses, sheep, goats, and swine), domestic animals (e.g., rabbits, dogs, and cats), and laboratory animals (e.g., rats, mice, and guinea pigs). Examples of non-mammals include, but are not limited to, birds. The term “subject” does not denote a particular age or sex. In one embodiment, the subject is a human. In a particular embodiment, the human is a human suffering from a vascular disease or dysfunction, or a disease having associated vascular dysfunction, e.g., type 2 diabetes.

As used herein, the terms “administering” and “administration” refer to any method of providing a pharmaceutical preparation or composition to a subject comprising the Fis1 inhibitory peptides described herein. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, intradermal administration, intrathecal administration and subcutaneous administration. Administration can be continuous or intermittent. In various aspects, a preparation can be administered therapeutically; that is, administered to treat an existing disease or condition.

To aid in administration, the peptides or vectors may be mixed with a suitable pharmaceutical acceptable carrier known in the art. The term “pharmaceutically acceptable” can refer to compositions approved by a regulatory agency (e.g., a federal or state government agency) for administration to a subject. The term “carrier” can refer to a diluent, excipient, or vehicle with which the pharmaceutical composition can be administered. Pharmaceutically acceptable carriers are known in the art and include, but are not limited to, for example, diluents, preservatives, solubilizers, emulsifiers, liposomes, nanoparticles among others. Examples of suitable pharmaceutical carriers are described in “Remington's Pharmaceutical Sciences” by E. W. Martin. Additionally, such pharmaceutically acceptable carriers may be solutions, suspensions, and emulsions in aqueous or non-aqueous solvents. Examples of nonaqueous solvents are propylene glycol, polyethylene glycol, vegetable oils such as olive oil, and injectable organic esters such as ethyl oleate. Suitable aqueous solvent carriers include isotonic solutions, alcoholic/aqueous solutions, emulsions or suspensions, including saline and buffered media. Such carriers include, but are not limited to, for example, water, oil (e.g., a vegetable oil), ethanol, saline solution (e.g., phosphate buffer saline or saline), aqueous dextrose (glucose) and related sugar solutions, glycerol, or a glycol such as propylene glycol or polyethylene glycol. Stabilizing agents, antioxidant agents and preservatives may also be added. Suitable antioxidant agents include sulfite, ascorbic acid, citric acid and its salts, and sodium EDTA. Suitable preservatives include benzalkonium chloride, methyl- or propyl-paraben, and chlorbutanol. The composition for parenteral administration may take the form of an aqueous or nonaqueous solution, dispersion, suspension or emulsion. The compositions may contain additional pharmaceutically acceptable substances as required to approximate physiological conditions such as a pH adjusting and buffering agent, toxicity adjusting agents, such as, sodium acetate, sodium chloride, potassium chloride, calcium chloride, sodium lactate, and the like.

The compositions can be sterilized by conventional, well-known sterilization techniques that maintain the activity of the peptide. The formulation should be selected according to the mode of administration. Buffers can include, but are not limited to, phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ brand surfactant, polyethylene glycol (PEG), and PLURONICS™ surfactant. Pharmaceutically acceptable carriers can include but are not limited to 0.01 to 0.1 M, preferably 0.05M, phosphate buffer or 0.9% saline. Other suitable pharmaceutically acceptable carriers are also contemplated.

For purposes of the present invention, “treating” or “treatment” describes the management and care of a subject for the purpose of combating the disease, condition, or disorder. Treating includes the administration of a peptide of present invention to reduce, inhibit or prevent the onset of the symptoms or complications, reduce or alleviating the symptoms or complications, or eliminating the disease, condition, or disorder. In a preferred embodiment, the disease is a vascular dysfunction. In a particular embodiment, the disease is type 2 diabetes with vascular dysfunction.

### Kits

Aspects of the disclosure described with respect to the former method can be applicable to the latter method and kits, and vice versa, unless the context clearly dictates otherwise.

Suitable kits are also included for preforming the methods described herein. Kits may comprises a Fis1 inhibitory peptide as described herein or compositions comprising the Fis1 mutant peptide and instructions for the use. In another embodiment, the kit may comprise the nucleic acid sequence encoding the Fis1 inhibitor peptide described herein or a cell capable of producing the Fis1 inhibitor peptide for production and purification. Instructions for production, purification or use may also be included within the kit. The kit may further comprise the pharmaceutical composition comprising the Fis1 inhibitory peptide described herein.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim. For example, with regard to sequences “consisting of” refers to the sequence listed in the SEQ ID NO. and does refer to larger sequences that may contain the SEQ ID as a portion thereof.

All publications, patent applications, patents, and other references mentioned herein are incorporated by reference in their entirety. In the case of conflict, the present specification, including definitions, will control.

Other features and advantages of the invention will be apparent from the description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting.

### EXAMPLES

### Example 1: Inhibition of Fis1 by Novel Peptide Pep213 or Molecular Suppression Reverses Diabetic- and High Glucose-Induced Endothelial Dysfunction in Human Resistance Arteries

Impaired vascular endothelial function predates the development of macrovascular and microvascular disease in patients with type 2 diabetes (T2DM). Emerging data implicate abnormal mitochondrial form and function in the development of vascular endothelial dysfunction in humans with T2DM.^{1,2} Mitochondria in the endothelium of individuals with T2DM produce excessive superoxide. The excess superoxide is driven, in part, by greater polarization of the mitochondrial inner membrane.² Excessive mitochondrial reactive oxygen species (mtROS) production in the endothelium activates critical epigenetic changes and cell signaling pathways that lead to endothelial inflammation and vascular dysfunction.³ We have previously shown that mitochondrial-targeted antioxidants or pharmacological agents dosed to partially depolarize the mitochondrial inner membrane reverse impaired endothelium-dependent vasodilation in resistance arterioles from humans with T2DM.^{2,4}

Unfortunately, the phase 3 clinical trials of anti-oxidant therapeutic approaches to prevent and treat vascular diseases have failed to verify positive effects seen in smaller physiological and/or non-randomized studies,^{5,6} and current pharmacological agents that target the mitochondrial inner membrane have toxicity profiles that preclude their clinical use.⁷

Targeting proteins involved in mitochondrial fission represent a promising alternative approach based on prior work demonstrating (1) increased expression of mitochondrial fission 1 protein (Fis1) in endothelial cells obtained from humans with type 2 diabetes; (2) molecular knockdown of Fis1 or dynamin-related protein 1 (Drp1, which may bind Fis1 to induce mitochondrial fission) expression blocks high-glucose induced increases in mitochondrial superoxide production and impairment of phosphorylation of endothelium-derived nitric oxide synthase (eNOS) at its Ser1177 activation site; and (3) pharmacological and molecular knockdown of Drp1 reverse low-glucose induced endothelial dysfunction in human resistance arterioles.^{1,8} Fis1 as a pharmacological target is of particular interest given its role in mitochondrial dynamics appears to be most highly active in the setting of pathological stimuli like hypoxemia and hyperglycemia.⁹⁻¹²

In this Example, we tested whether knockdown of Fis1 expression reversed impaired endothelium-dependent vasodilation and nitric oxide (NO) production in resistance vessels from patients with type 2 diabetes and in vessels from healthy individuals exposed acutely to high and low glucose concentrations. Additionally, we determined the effect of Fis1 knockdown on endothelial cell barrier function, oxygen consumption, and glycolysis under high or low glucose conditions. Finally, we designed and tested a novel peptide engineered to bind to Fis1 and block Fis-1 mediated fission (Pep213 (SEQ ID NO:1)) and demonstrated it favorably impacted endothelium-dependent vasodilation of small resistance arteries from humans with T2DM and from healthy human vessels exposed to high glucose concentrations. Our findings support our contention that pharmacological targeting of Fis1 provides a useful therapeutic avenue for the challenge of vascular disease in T2DM.

Summary of Results: Fis1 knockdown improved endothelium dependent vasodilation in T2DM arterioles (P=0.002), and blocked HG (P=0.0008) and LG-induced (P=0.0002) impairment of endothelium-dependent vasodilation of healthy vessels. Fis1 knockdown preserved NO bioavailability and improved endothelial layer integrity of cells exposed to HG or LG (P<0.001). Fis1 knockdown had no significant effect on the expression of other mitochondrial dynamics or autophagy proteins, and had no effect on endothelial cell metabolism. Pep213 demonstrated low micromolar affinity for Fis1 (3.3-7 μM). Tat sequence-linked pep213 improved endothelium-dependent vasodilation in T2DM (P<0.001) and non-T2DM vessels exposed to HG (P<0.001).

### Methods:

Subject Recruitment and Screening: We recruited 67 individuals with T2DM and healthy individuals (ages 21-75) without cardiovascular risk factors as previously described.^{2,13} Individuals with T2DM were either on medications to treat T2DM and/or met criteria for the diagnosis of T2DM American Diabetes Association.¹⁴ Study methods were reviewed and approved by the Institutional Research Board of the Medical College of Wisconsin and all subjects signed a written informed consent form prior to proceeding with any study activities. All subjects were screened to assure they met study inclusion criteria. In all subjects, height and weight were measured and heart rates and blood pressures measured in triplicate. Subjects with or without T2DM were excluded if they had any of the following: known atherosclerotic disease (coronary artery disease, peripheral vascular disease, history of stroke or myocardial infarction), chronic liver disease, elevated plasma creatinine (>1.5 mg/dl in men, >1.4 mg/dl in women), a diagnosis of cancer in remission less than a year, regularly taking a blood thinner or anti-platelet agent other than aspirin, or smoking cigarettes within a year of enrollment. Individuals without T2DM were also excluded if they had an LDL cholesterol≥160 mg/dl or hypertension (blood pressure≥140/90 mmHg) or if they were on medications to treat either of these entities. Vessels for the studies comparing the impact of pep213-tat and scrambled control peptides on endothelium dependent vasodilation (N=5 non-T2DM, N=4 T2DM) were obtained from discarded subcutaneous adipose tissue under a separate protocol reviewed and approved by the Medical College of Wisconsin's Institutional Research Board.

Human Resistance Artery Acquisition: Human resistance arteries from adipose samples were obtained from the upper outer quadrant of the gluteal adipose pad as previously described.^{2,8,13,15,16} Briefly, following sterilization and anesthesia with 1% lidocaine, a 1-1.5 cm incision is made in the upper outer quadrant of the gluteal adipose pad. An approximately 8 cm³ volume of adipose tissue was removed by sharp dissection. After achieving hemostasis, the wounds were closed with 1-2 absorbable deep dermal sutures and the epidermal layer closed using either Dermabond or steristrips.

Cell Culture: Immortalized human microvascular endothelial cells (HMEC-1) purchased from ATCC (Manassas, VA) were cultured in antibiotic-free MCDB131 (Life Technologies, Carlsbad, CA) supplemented with 10 mM Glutamine (Thermo Fisher Scientific, Waltham, MA), 10% FBS (Sigma-Aldrich, St. Louis, MO), 10 ng/mL Human EGF (Thermo Fisher Scientific), and 1 μg/mL Hydrocortisone (Sigma-Aldrich). For assays involving high glucose (33 mM), normal glucose (5 mM), or low glucose (2.5 mM) conditions, antibiotic-free MCDB131 was supplemented with the above mentioned and glucose levels were adjusted by adding glucose and sterile PBS. Human dermal microvascular endothelial cells (HMEC) were purchased from Lonza (Basel, Switzerland) and supplemented with Microvascular Endothelial Cell Growth Medium-2 Bullet Kit (Lonza).

### Transfection Protocols for Cultured Endothelial Cells and Human Vessels

Transfection of Fis1 and Drp1 siRNA and Scrambled Control into Cultured Cells Constructs for Fis1 and Drp1 siRNAs were acquired from Origene (Rockville, MD, sequences below). Lipofectamine RNAi Max (Thermo Fisher Scientific) vehicle was added to 20 nM of RNAi constructs in Opti-MEM (Life Technologies). The mixture was diluted in culture media and incubated with HMEC-1 cells for four hours prior replacement with normal culture media. Cells were subsequently incubated for 24 hours before treatment and analyses. Following incubation, cells treated exposed to either high glucose for 6 hours or low glucose for 2 hours prior to measuring NO production, bioenergetics, protein expression, and endothelial layer integrity.
Transfection of Fis1 and Drp1 siRNA and Scrambled Controls in Human Resistance Arteries

Transfection of human resistance arteries with siRNA constructs was performed as previously described.^{8,13} Briefly, resistance arteries dissected from adipose tissue were suspended in a culture myograph chamber (204 CM, DMT, Ann Arbor). One end of the vessel was sutured onto a glass pipette within a micro-organ chamber. Prior to suturing the second end of the arteriole to a glass pipette, Fis1 siRNA, Drp1 siRNA (20 nM in optiMEM using Lipofectamine RNAiMax, Invitrogen) or control scrambled siRNA (20 nM) were gently injected, into the vessel lumen. The loose end of the vessel is then tied onto another glass pipette, and the vessel is suspended in the chamber and placed on a myograph stage bathed in physiological buffer at 37° C. and pressurized to 60 mmHg. Following 4-6 hours of incubation, the siRNA is slowly washed out of the lumen over 24 hours at a low shear rate (□<5 dyn/cm²).

Measurement of Endothelium-Dependent Vasoactivity: Transfected resistance arteries from healthy individuals were exposed to either normal glucose conditions (NG, glucose level 5 mM), 2 hours of low glucose (LG, glucose level 2.5 mM) or 6 hours of high glucose (HG, glucose level 33 mM). All studies in vessels from patients with T2DM were performed at 5 mM glucose. Vessels were pre-constricted approximately 50% of resting diameter with endothelin-1 (Sigma Aldrich, USA). The constricted arterioles are then exposed to acetylcholine (Ach) at serially increasing doses from 10⁻¹⁰ to 10⁻⁵ M, and changes in vessel diameter were measured using digital calipers and videomicroscopy. Following the 10⁻⁵ M dose, vessels were exposed to 200 μM papaverine to test the smooth muscle reactivity. Following a 30-minute washout period, the same arterioles were re-constricted and incubated with L-NG-nitroarginine methyl ester (L-NAME, 100 μM), a direct inhibitor of nitric oxide synthase, for 30 minutes and vasodilation subsequently was remeasured following the exposure to Ach 10⁻¹⁰ to 10⁻⁵ M.

### Measurements of Nitric Oxide (NO) Bioavailability:

Cells in Culture: To measure nitric oxide (NO) bioavailability in HMEC-1 cells and arterioles, a fluorescent NO marker, 4,5-diaminofluorescein diacetate (DAF2-DA from Cayman Chemical) was used. In a dark room, cells were incubated with or without 100 μM L-NAME for 2 hours followed by 15 minutes of incubation with DAF2-DA (5 μM) at 37° C. Fluorescence intensity was measured using a SPECTRAFluor Plus plate reader (Tecan, Morrisville, NC) using excitation and emission wavelengths of 485 nm and 535 nm, respectively.
Human Resistance Arteries: Two vessels were obtained from a subset of healthy subjects and each vessel was cut in half to allow for four experimental conditions which included transfection with Fis1 siRNA or Drp1 siRNA with and without concomitant exposure to L-NAME for 30 minutes and transfection with scrambled siRNA control with and without concomitant exposure to L-NAME at room temperature. Ach 10⁻⁵ and DAF2-DA (5 μM final concentration) were subsequently added to each vessel and incubated at 37° C. for 30 minutes. The arterioles were then washed with PBS buffer, mounted on a slide, and imaged by fluorescence microscopy. Untreated and unstained arterioles were used as controls for fluorescence background interference. Both treated and control vessels were measured using the same gain settings and the results were analyzed using Meta Morph 7.8 software (Universal imaging, West Chester, PA).

### Measurement of Endothelial Layer Integrity:

Human microvascular endothelial (IMEC-1) cells were seeded at 40,000 cells/well and grown on gold electrode array plate (8W10E+, Applied Biophysics Inc.) until 50% confluent. The cells were pre-transfected with Fis-1 siRNA (20 nM) or scrambled siRNA (20 nM). On the days of experiments, the transfected cells were exposed to different glucose conditions: high glucose (33 mM) for 6 hours, normal glucose (5 mM) for 2 hours and low glucose (2.5 mM) for 2 hours. The integrity of the monolayers were checked at 64,000 Hz with less than 10 nF capacitance. The cells were then subjected to Electric Cell-substrate Impedance Sensing (ECIS) functional assay, and monolayer transepithelial electrical resistance (TEER) was measured in real time using an ECIS ZTheta Instrument (Applied Biophysics Inc., Troy, NY). Resistance was measured over the following frequencies, with 4,000 Hz as the standard: 125 Hz, 250 Hz, 500 Hz, 1,000 Hz, 2,000 Hz, 4,000 Hz, 8,000 Hz, 16,000 Hz, 32,000 Hz, and 64,000 Hz.

### Mitochondrial Bioenergetic Measurements:

The XFe96 Analyzer (Seahorse Biosciences, North Billerica, MA, USA) was used to measure mitochondrial bioenergetics by measuring the oxygen consumption rate (OCR) and extracellular acidification rate (ECAR). HMEC-1 cells were seeded (20,000 cells/well) onto a 96-well Seahorse microplate 4-6 hrs. before treatment to let the cells adhere to the plate. Cells were pre-transfected with siRNA targeting Fis1 and siRNA scrambled control. The medium was aspirated and replaced with either high glucose for 6 hours or normal glucose or low glucose for 2 hours. The high, normal, or low glucose medium was removed and replaced with XF Base Medium Minimal Dulbecco's modified Eagle's medium (DMEM, pH 7.40) supplemented with 600 μL glucose 45%, 1.5 mL L-Glutamine (200 mM), and 1.5 mL sodium pyruvate (100 mM) with the final glucose concentration the same as the initial medium concentration. Cells were then incubated in a CO₂-free incubator at 37° C. for 1 hour for temperature and pH calibration. During mitochondrial stress tests, the following drugs were sequentially injected in increments of 25 μL: Oligomycin A (2.5 μM final well concentration), FCCP (carbonyl cyanide 4-(trifluoromethoxy) phenylhydrazone, 1 μM final well concentration), and rotenone & antimycin A (1 μM final well concentration each). During glycolytic stress tests the following substances were sequentially injected in increments of 25 μL: glucose (10 mM final well concentration), oligomycin A (2.5 μM final well concentration), and 2-deoxyglucose (50 mM final well concentration).

Following these measurements, cells were fixed with 4% paraformaldehyde for 15 minutes and stained with DAPI (4′,6-diamidino-2-phenylindole) at 1:500 of 1.5 μg/mL for 24 hrs. Cell counts were obtained using automated cell counting on Cytation5 cell imaging multi-mode reader (BioTek, Vermont, USA) with OCR and ECAR measurements then normalized to cell number.

### Measurement of Mitochondrial Proteins:

HMEC-1 cells were grown on a six well plates at 0.3×10⁶ cells/well and transfected with siFis1 and siRNA. After transfecting for 4-6 hours, the medium was changed to normal cell culture medium and incubated overnight. Cells were treated either with HG (33 mM) for 6 hours, or NG (5 mM) or LG (2.5 mM) for 2 hours. The plates were then washed 2× with cell wash buffer and 50 μl RIPA lysis buffer (ProteinSimple, San Jose, CA) was added to each well. The cells were scraped gently on ice and transferred to a labelled tube. Lysates were centrifuged at 10,000 rpm for 10 minutes and snap frozen in liquid nitrogen and kept at −80° C. overnight. The cell pellet and supernatant were thawed, vortexed briefly, and centrifuged at 10,000 rpm for 10 minutes. The supernatant was transferred to labelled tubes, protein concentration quantified by Bradford assay, and protein expression evaluated by automated capillary electrophoresis-based immunodetection via WES (ProteinSimple). Proteins were analyzed and detected with the 12-230 kDa WES separation module and 25 capillary cartridges. Lysates were diluted in 0.1 X sample buffer in a 4:1 ratio of sample to fluorescent master mix, then denatured at 95° C. for 5 min. For immunodetection, blocking buffer, primary antibody, secondary antibody, chemiluminescent substrate, samples, a biotinylated size marker and wash buffer were loaded in designated wells on the supplied microplate. The plate was centrifuged 5 min at 1000 X g then loaded into WES. Protein detection used default separation parameters. Compass for SW software (version 3.1.7, ProteinSimple) analyzed data and integrated specific antibody peaks. Across samples, protein expression was normalized to total protein. Total protein was analyzed in an addition WES assay with biotin-based protein labeling. Total protein was also analyzed using Compass for SW software as the integral of the antibody peaks. Control carryover samples between plates were used to correct for inter-plate signal intensity variability. The average quotient of antibody peak integrals with sample over total protein was taken.

### NAMR Titration Experiments:

To determine peptide binding affinities for Fis1, NMR titration experiments were performed similarly to chemical fragment titrations as described.{Egner, 2018 #10126} First, peptides were resuspended in Fis1 dialysate buffer (100 mM HEPES pH 7.4, 200 mM NaCl, 1 mM DTT, 0.02% v/v sodium azide) to a final concentration of 6 mM. Then, 220 μL of 50 μM ¹⁵N-hFis1 and increasing amounts of peptide (0, 25, 50, 150, 400, 800, 1600, and 2000 μM) was prepared and loaded into 3 mm NMR tubes. For each sample, ¹H, ¹⁵N HSQC spectra were collected at 25° C. on a Bruker Advance II 600 MHz spectrometer equipped with a triple resonance z-axis gradient cryoprobe and SampleJet autosampler, which allowed automatic tuning, shimming, and data collection for each sample. ¹H, ¹⁵N HSQC experiments consisted of 8 scans with 1024 and 300 complex points in the ¹H and ¹⁵N dimensions, respectively. Spectra were processed with automated python scripts using NMRPipe and chemical shifts were measured using TitrView and CARA software.{Delaglio, 1995 #10127}{Masse, 2005 #10128} Peptide binding affinity was determined by TREND analysis to each spectrum within the titration series. {Xu, 2016 #10129}{Xu, 2017 #10131} TREND reveals the changes in data by performing principal component analysis, where each concentration point is treated as a unique data point; here, the input is each ¹H, ¹⁵N spectrum with increasing amounts of peptide. After performing principal component analysis, the principal component 1 (PC1) values are normalized to the highest PC1 value resulting in a range from 0 to 1.

Then, the normalized PC1 values are plotted against peptide concentration and fit to a ligand depletion function with protein concentration held constant (Eq. 1)

$$Δ = \frac{{d}_{max} * ( ( {K}_{d} + p + l ) - {( {( {K}_{d} + p + l )}^{2} - 4 * p * l )}^{0.5} )}{2 * p} , \qquad Equation 1$$

where Δ=adjusted chemical shift change, □ₘₐₓ=maximum chemical shift perturbation, K_{d}=binding dissociation constant, p=[protein], and l=[ligand].

Spectral overlays were generated using XEASY software and Adobe Illustrator.

Synthesis of pep213:

Peptides pep213 (SHKHDPLPYPHFLL, SEQ ID NO:1) and TAT-p213 (YGRKKRRQRRRGSGSGSSHKHDPLPYPHFLL, SEQ ID NO:3) were all purchased from Genscript (Piscataway, NJ) with N-terminal acetylation and C-terminal amidation and >95% purity by HPLC. The TAT-p213 fusion peptide included a GSGSGS (SEQ ID NO:4) linker between the TAT cell penetrating sequence (YGRKKRRQRRR, SEQ ID NO:2) and pep213.

### Intrinsic Tryptophan Fluorescence:

Tryptophan fluorescence data was collected on a PTI Model #814 fluorimeter using a 2ex of 295 nm and λₑₘ of 300-400 nm within Starna Cells 3-Q-10 quartz fluorometer rectangular cell with a 10 mm pathlength and excitation/emission slit widths of 4/6 nm, respectively. A concentrated stock of peptide (pep213) was resuspended in final hFis1 dialysate buffer and a concentration series with the following points was prepared: 0, 1, 3, 7, 10, 30, 70, 100, 300, 700, and 1000 μM peptide. For each titration point, tryptophan emission spectra were collected on samples excluding Fis1 and then, 5 μL of 400 μM hFis1 was added to the sample for a final concentration of 10 μM hFis1 and tryptophan emission spectra were recollected. To account for buffer and tyrosine fluorescence background from the peptide, difference emission spectra were generated by subtracting the background fluorescence intensities from spectra lacking Fis1. The average emission wavelength at each peptide concentration was calculated according to Equation 3 and plotted as a function of the natural log of peptide concentration, which was fit to a Boltzmann sigmoidal model (Equation 4). {Royer 1993 #10132}

### Equation 3:

$$λ = \frac{{\sum }_{n = 1}^{N} {I}_{n} * {λ}_{n}}{{\sum }_{n = 1}^{N} {I}_{n}} ,$$

where [symbol] λ [symbol] =average emission wavelength, Iₙ=fluorescence intensity emitted at wavelength λₙ, and the summation calculated from λₙ of 310 to 370 nm.

$$λ = A + \frac{( B - A )}{1 + {e}^{\frac{{K}_{D - p}}{c}}} , \qquad Equation 4$$

where λ=average emission wavelength, A=pre-transition phase, B=post-transition phase, K_{D}=equilibrium dissociation constant, p=natural log of the peptide concentration, and c=slope of the transition phase.
Impact of pep213 on Endothelial Cell NO Bioavailability and Endothelium-Dependent Vasodilation: Vessels from a subset of subjects with and without T2DM were selected at random for these studies. Vessels from healthy subjects were pre-treated with HG (33 mM) for six hours and subsequently exposed to 1 or 10 μM of pep213 attached to a TAT sequence to facilitate cellular uptake. Vessels from T2DM subjects were incubated under NG conditions (5 mM) and exposed to either 1 or 10 μM of pep213-TAT. Endothelium-dependent vasodilation with increasing doses of acetylcholine, smooth muscle reactivity to papaverine, and eNOS-dependence of the vasodilatory response to acetylcholine using L-NAME were assessed as described previously.^{2,4,8,15}

### Statistical Analyses:

Statistical analyses were performed with either GraphPad Prism V7.03 (GraphPad Prism version 8.0.0 for Windows, GraphPad Software, San Diego, California, USA) or SigmaPlot Version 12.5 (Systat Software, San Jose, California, USA). P<0.05 were considered statistically significant. Data are presented as mean±SE unless otherwise stated. All functional vessel data was analyzed by two-way ANOVA with post-hoc testing to determine the differences between groups (Dunnett's multiple comparison test) and dose-response (Tukey's multiple comparison tests). The TEER assay data and bioenergetics data were also analyzed by two-way ANOVA followed by post-hoc testing (Tukey's multiple comparison tests) to determine the differences between HG, LG, and NG treatments. DAF2-DA fluorescence intensity in human vessels, Fis1 knockdown-efficiency in HMEC-1 cells, and measurements of mitochondrial protein amounts by Western blot were analyzed by one-way ANOVA followed by Tukey's multiple comparisons to assess differences between groups. DAF2-DA fluorescence intensity in HMEC-1 cells transfected with siFis1 and scrambled siRNA were analyzed by two-way ANOVA followed by post-hoc testing (Tukey's multiple comparison tests). NO production and relative ratio of P-eNOS and β-actin in HMEC-1 cells stimulated with A23187 were analyzed using paired Students t-test.

### Detailed Results:

Study Subject Characteristics: A total of 67 subjects (14 T2DM, 53 healthy controls) were recruited. A full description of the characteristics of subjects who participated in this project's studies is presented in Table 1. Our healthy subjects were significantly younger than the subjects with T2DM (39±15 vs. 55±12 years, P=0.0002). Our healthy control subject group had a significantly lower body mass index, waist circumference, systolic blood pressure, fasting glucose, and glycosylated hemoglobin. Healthy subjects had higher HDL cholesterol levels as well. None of the healthy subjects were taking chronic cardiometabolic medications.

We additionally present the subject characteristics for each investigation involving humans vessels in Supplemental Table 1 (subjects whose vessels were transfected with Fis1 siRNA or scrambled control and endothelium-dependent vasodilation tested), Supplemental Table 2 (subjects whose vessels were transfected with Fis1 siRNA or scrambled control and NO bioavailability tested), Supplemental Table 3 (subjects whose vessels were transfected with Drp1 siRNA or scrambled control and endothelium-dependent vasodilation tested), Supplemental Table 4 (subjects whose vessels were transfected with Fis1 siRNA or scrambled control and NO bioavailability tested), and Supplemental Table 5 (subjects whose vessels were exposed to pep213 and vasoactivity tested).

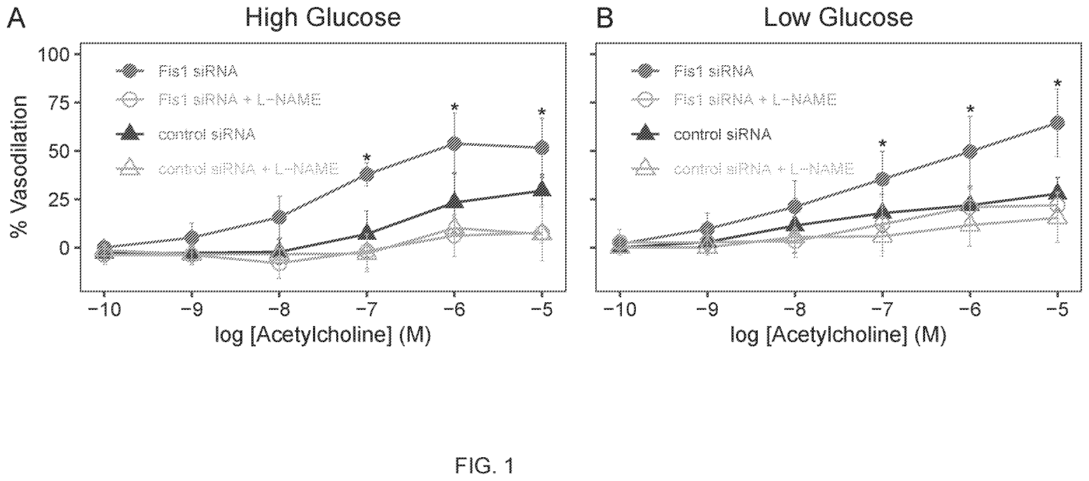

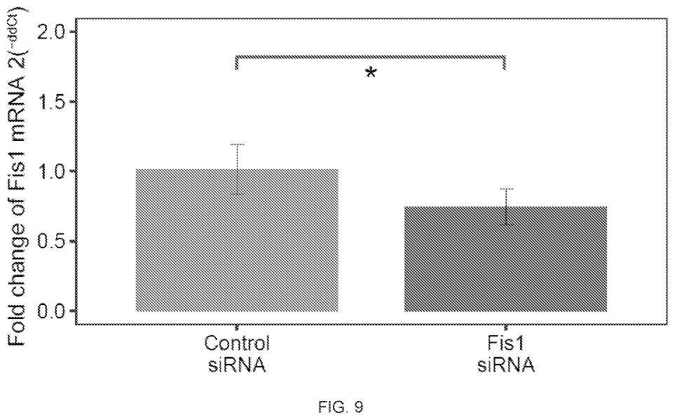

Impact of Suppression Fis1 or Drp1 on Endothelium-Dependent Vasodilation and NO Bioavailability in Human Vessels: Using our protocol, we measured vasodilation in healthy human resistance vessels incubated with glucose at concentrations experienced by T2DM patients (FIG. 1A-B). Consistent with our earlier findings, both high (33 mM) and low (2.5 mM) glucose blunted the endothelium-dependent vasodilatory response to acetylcholine that appeared primary related to eNOS activity based on inhibition with L-NAME. To address whether the mitochondrial fission protein Fis1 might be involved in this impairment, we transfected the healthy vessels with Fis1 siRNA that leads to approximately 30% reduction in Fis1 mRNA (FIG. 9). In these healthy human resistance vessels, transfection of Fis1 siRNA protected against HG and LG-induced impairments in endothelium-dependent vasodilation (FIG. 1A-B). Administration of L-NAME completely blocked this protective effect in both cases suggesting that Fis1 siRNA associated improvements are eNOS-dependent.

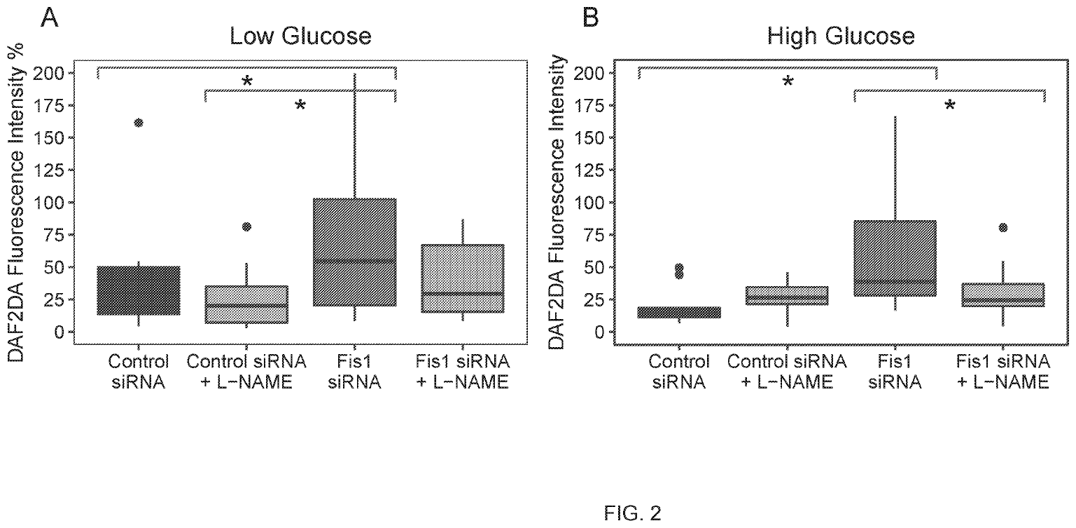

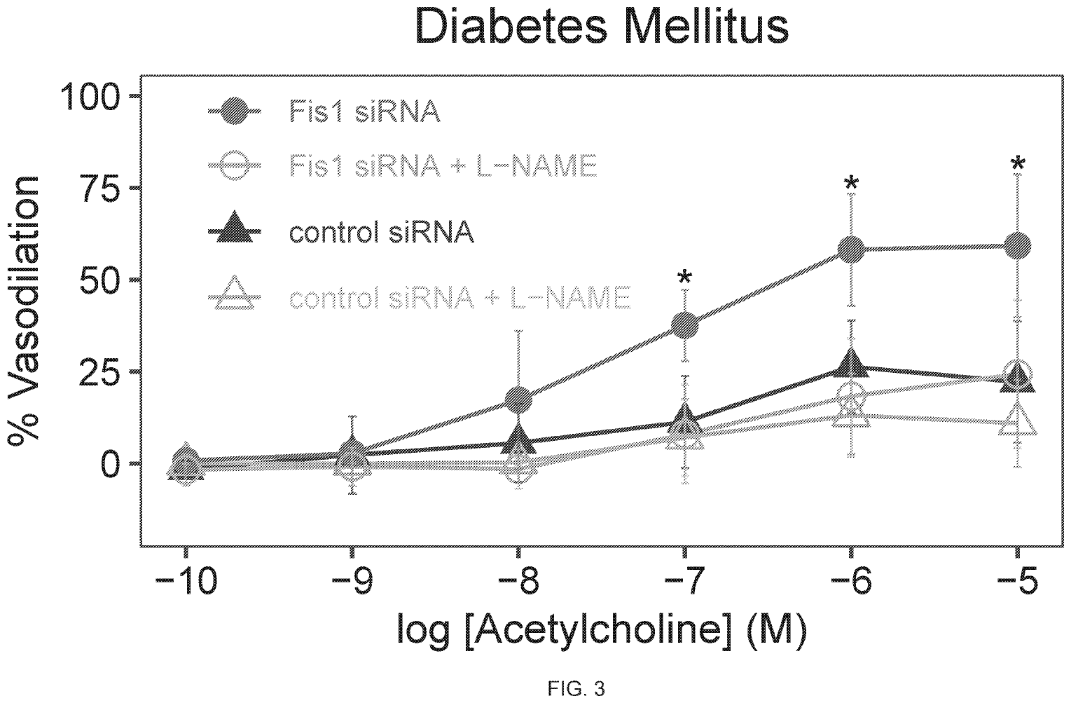

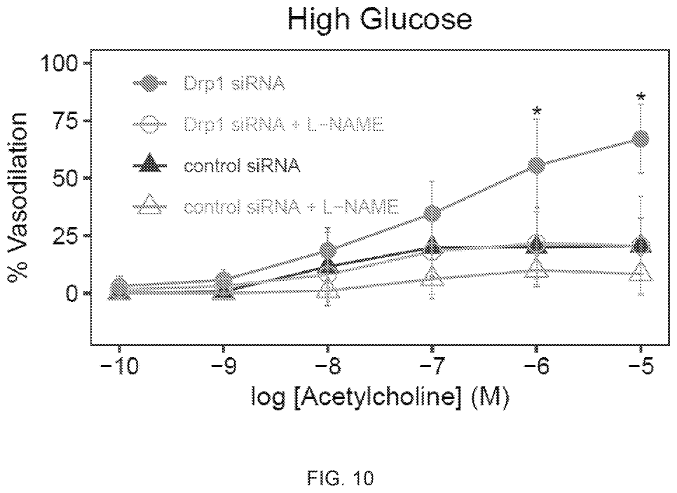

Consistent with this interpretation, Fis1 siRNA transfection also significantly increased the fluorescence of NO-sensitive dye DAF2-DA in vessels from healthy individuals exposed to HG [FIG. 2A; n=9, P=0.04 for Fis1 siRNA versus all other exposures (scrambled control, scrambled control+LNAME, and Fis1 siRNA+L-NAME)] and LG (FIG. 2B, n=8, P=0.01 for Fis1 siRNA vs. all other exposures). L-NAME completely abolished the increased in DAF2-DA fluorescence in both cases. These data support the concept that Fis1 silencing can improve vasodilation in an NO-dependent manner under stress conditions experienced by T2DM patients. We next asked whether Fis1 siRNA treatment could improve vasodilatory activity in resistance vessels from T2DM patients. Treatment with Fis1 siRNA reversed impaired endothelium-dependent vasodilation (FIG. 3; n=6, P=0.002 overall). As with vessels from healthy subjects, the favorable effect of Fis1 siRNA transfection on vessels from diabetic subjects was completely attenuated by L-NAME. Fis1 siRNA transfection did not impact the papaverine effect on vasodilation in any of these studies (data not shown). We had similar findings with genetic silencing of Drp1 in vessels from healthy and diabetic individuals (FIG. 10-12) consistent with excessive mitochondrial fission playing an important role in impairing NO-dependent vasodilation in the diabetic endothelium.

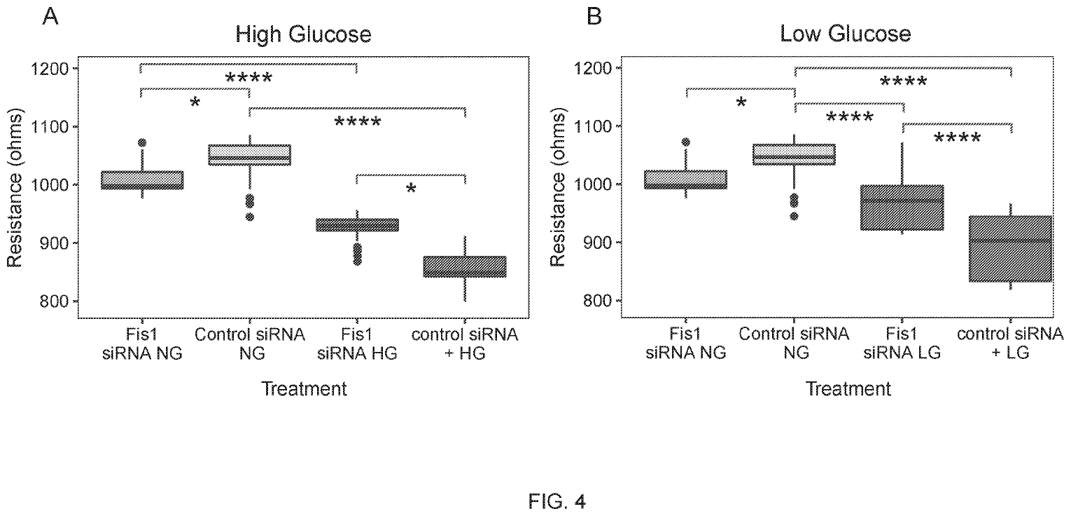

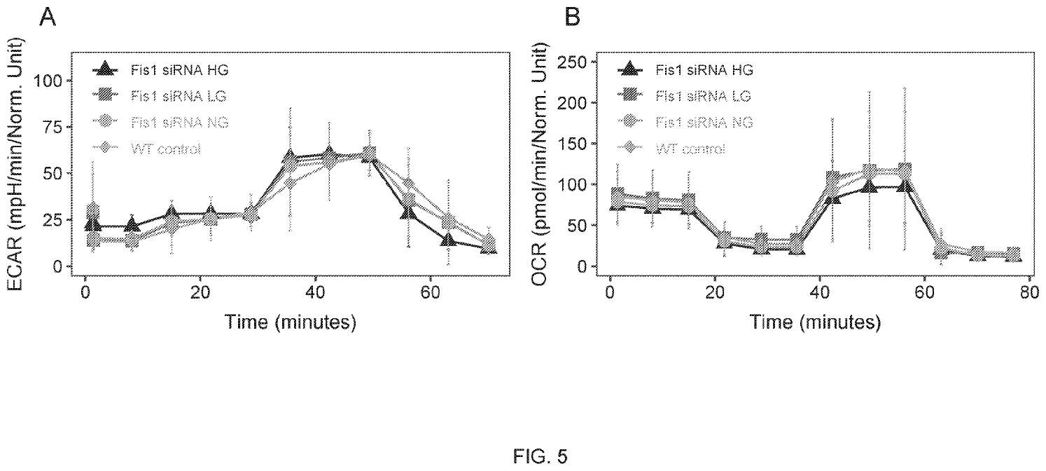

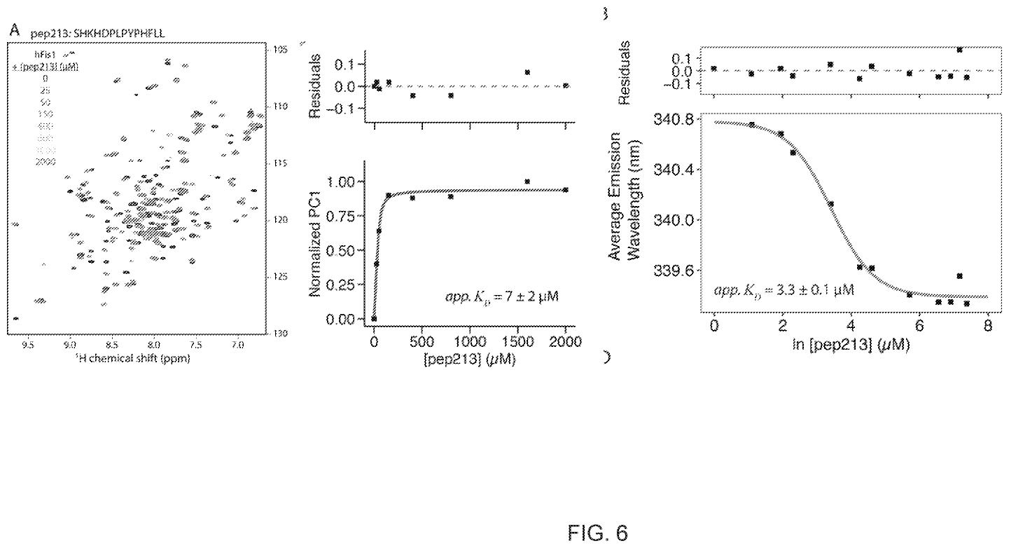

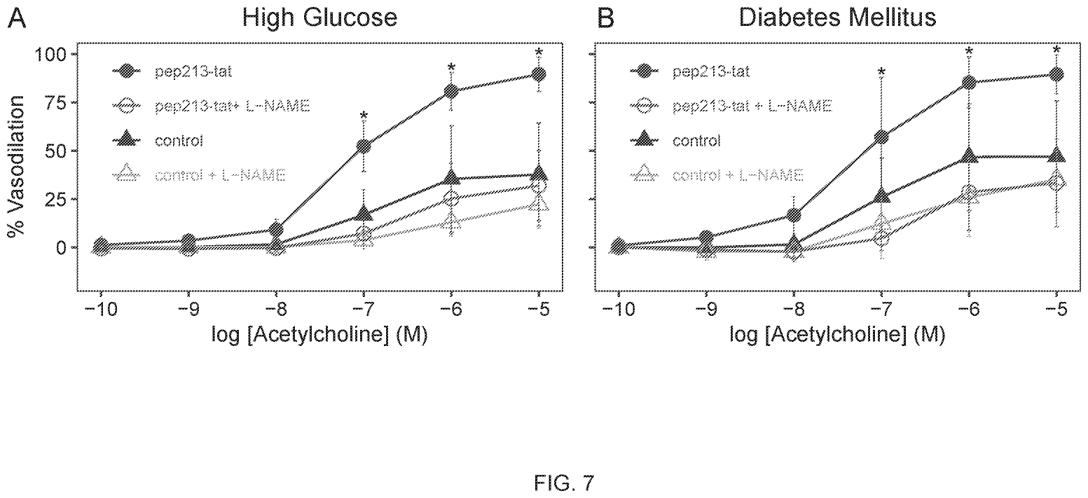

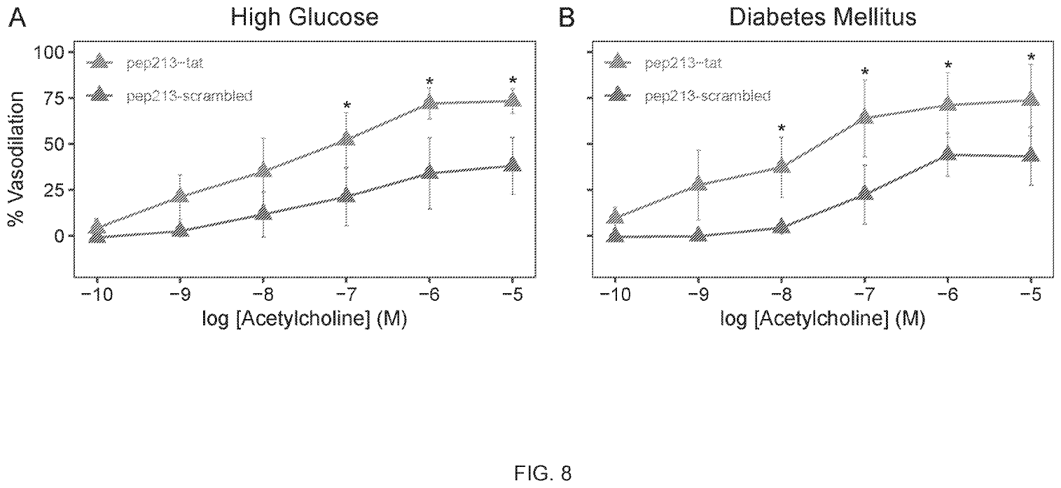

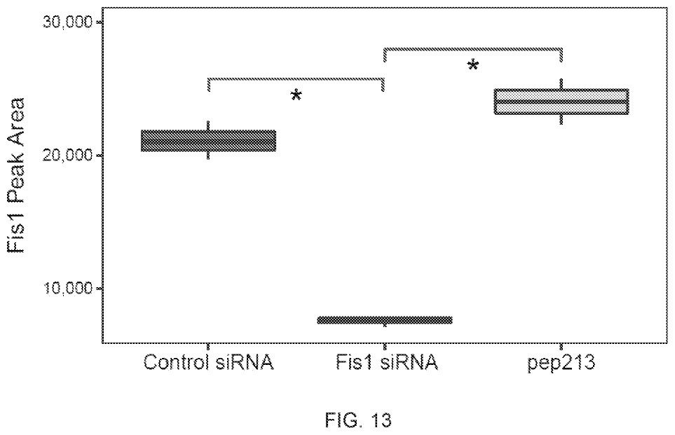

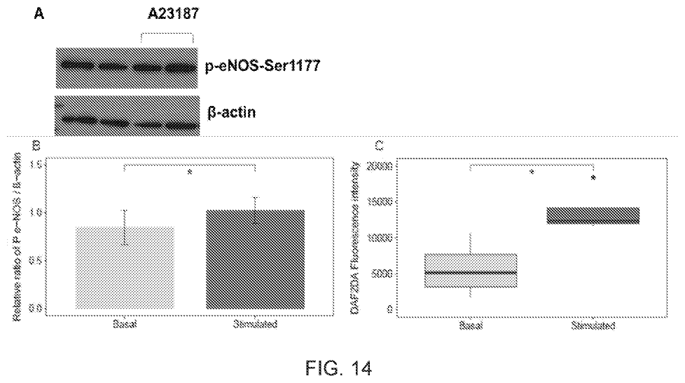

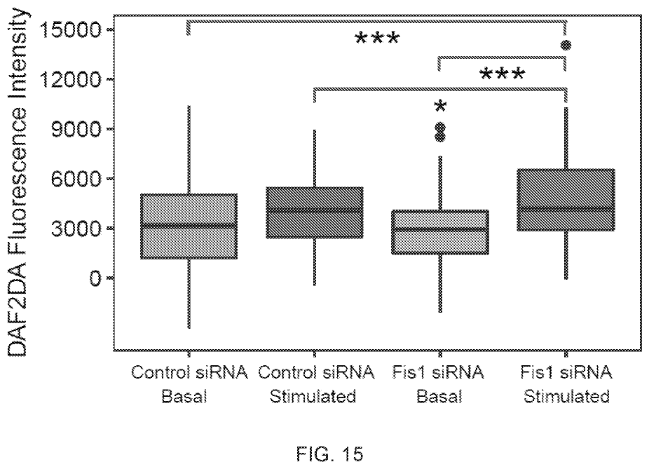

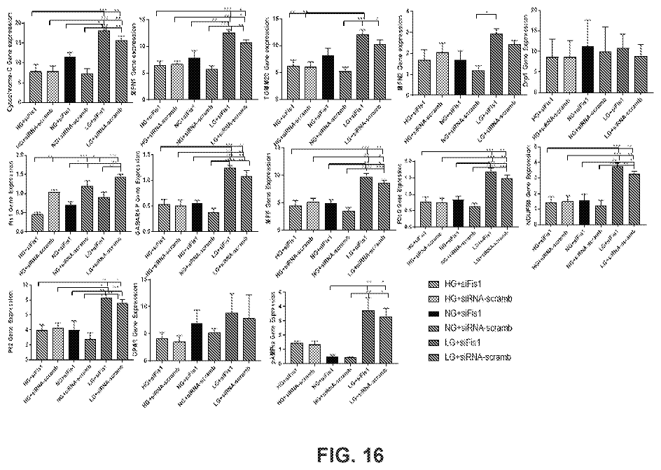

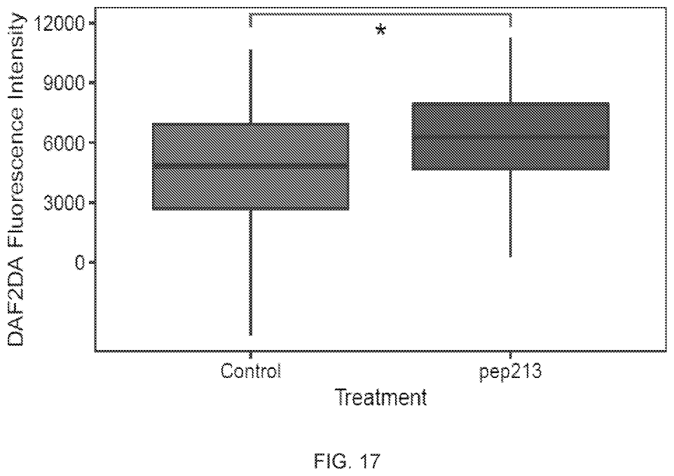

Impact of Fis1 Transfection on Fis1 expression, NO production, eNOS activation, and Expression of Mitochondrial Protein Associated with Fission, Fusion, and Autophagy: HMEC-1 cells transfected with Fis1 siRNA showed a significant 70% reduction in Fis1 expression (FIG. 13). Confirming the responsiveness of HMEC-1 to stimulus that increases eNOS activation and NO bioavailability, calcium ionophore A23187 drove a significant increase in both eNOS phosphorylation at the Ser1177 activation site and DAF2-DA fluorescence intensity in non-transfected HMEC-1 cells (FIG. 14A, N=6, P=0.03; 14B, N=4, P=0.02). Fis1 siRNA transfection also significantly increased DAF2-DA signal from HMEC-1 cells stimulated with A23187 compared to transfection of scrambled control siRNA transfection (FIG. 15, N=7, P<0.0001). Knocking down Fis1 expression did not significantly change expression of mitochondrial proteins involved in mitochondrial fission, fusion, and autophagy including cytochrome c, MFN1, MFN2, Drp1, GABARAP, MFF, POLG, NDUF88, P62, OPA1, or AMP Kinase (FIG. 16). The lack of impact on expression of these proteins was apparent under HG, LG, and NG incubation conditions. High and low glucose did influence the expression of some of these proteins but Fis1 knockdown had no effect within a given glucose exposure.
Impact of Fis1 Knockdown and Endothelial Cell Layer Integrity: The impact of Fis1 suppression on endothelial cell layer integrity under HG and NG conditions is reported in FIG. 4A. Under NG conditions, knockdown of Fis1 expression modestly but statistically significantly reduced endothelial cell layer resistance (P<0.05). Under HG conditions, endothelial cell layer resistance was significantly lower than resistance under NG conditions with or without Fis1 knockdown (P<0.001). Fis1 knockdown significantly increased resistance under HG conditions (P<0.0001) but remained lower than the resistance measured under NG conditions. Similar findings were found for LG (FIG. 4B).
Impact of Fis1 Knockdown on Oxygen Consumption and Glycolysis: Knockdown of Fis1 had no effect on either the extracellular acidification rate or oxygen consumption regardless of glucose exposure (NG, HG, and LG; n=5 for all measurements, FIG. 5) suggesting that inhibition of Fis1 does not significantly impact mitochondrial bioenergetics.
Determination of the Affinity of pep213for Fis1: Previous work identified several potential Fis1 binding peptides by phage display screening against a truncated version of the protein.¹⁷ We tested a subset of these peptides for binding to Fis1 and found weak affinity (high μM). From these data, we designed a novel peptide, pep213 (SEQ ID NO:1), that we reasoned might have higher affinity to Fis1 and block its activity. To evaluate this, we titrated increasing amount of the peptide into an NMR sample of Fis1 uniformly labeled with the stable isotope ¹⁵N. A superposition of the resulting ¹H-¹⁵N HSQC spectra shows many signals change upon addition of pep213 indicating binding (FIG. 6A). The resulting data can be globally fit to a single site binding model and gave an apparent K_{D}=7±2 μM (FIG. 6B). This affinity was confirmed by tryptophan fluorescence experiments in which changes in the tryptophan emission spectra upon addition of pep213 were fit to a binding isotherm to give a similar apparent K_{D}=3.3±0.1 μM (FIG. 6C). Another peptide (pep213-scrambled) consisting of the same amino acid composition as pep213, but in random order, showed no chemical shift perturbations upon addition to ¹⁵N-Fis1 at 2 mM indicating the Fis1-pep213 interaction is specific (FIG. 6D).
Impact of pep213 on Endothelium-Dependent Vasodilation in Human Resistance Vessels: Exposure to pep213 attached to TAT peptide to facilitate cellular uptake resulted in a significant improvement in endothelium-dependent vasodilation in both vessels from healthy subjects exposed to high glucose (FIG. 7A, N=6, P<0.0001 overall) and vessels from T2DM subjects (FIG. 7B, N=4, P<0.05 overall). In both cases, treatment with L-NAME abolished the improvements seen with pep213-TAT. While pep213-TAT reversed high-glucose induced impairment of endothelium-dependent vasodilation and impaired endothelium-dependent vasodilation in vessels from subjects with T2DM, a scrambled peptide using the same amino acids in a random sequence had no effect on either set of vessels (FIG. 8 N=5, P<0.001 overall). No differences were seen in the papaverine responses (data not shown) in any of these studies suggesting pep213-TAT did not impact smooth muscle reactivity. Additionally, human microvascular endothelial cells exposed to pep213-TAT for one hour showed a significant increase in DAF2-DA fluorescence (FIG. 17, N=3, P=0.04).

### Discussion:

These investigations reveal several novel findings. First, molecular inhibition of Fis1 blocks the unfavorable impacts of high- and low-glucose exposures on the ability human microvascular endothelial cells to produce NO, vasodilate in an endothelium-dependent manner, and maintain the endothelial cell layer barrier function. Additionally, molecular inhibition of Fis1 expression reverses impairment of endothelium-dependent vasodilation in human vessels from T2DM patients. These favorable effects occur without changes in endothelial cell metabolism or the expression of other proteins involved in mitochondrial fusion, fission, or autophagy. Additionally, using our knowledge of the structure of a critical binding site on Fis1, we designed a peptide with low micromolar binding affinity to Fis1 at this site, pep213, and verified its binding using two independent methods. Finally, we showed that pep213 is biologically active, increases NO bioavailability in human microvascular endothelial cells, and reverses high glucose-induced and type 2 diabetes-associated impairment of endothelium-dependent vasodilation in human resistance arteries in a nitric oxide synthase-dependent manner. These findings suggest a critical mechanistic role for excessive Fis1 expression and activity in the impairment of endothelial function in the settings of both acute and chronically abnormal glucose levels. Additionally, these data support the concept that a pharmacological therapy targeting to Fis1 holds promise for improving vascular health in patients with type 2 diabetes.

A strong rationale exists for exploring whether targeting mitochondrial proteins involved regulating the mitochondrial dynamics would lead to a favorable impact on human vascular endothelial function in patients with type 2 diabetes or patients exposed to high glucose concentrations. Properly balanced mitochondrial network dynamics are critical for maintaining normal mitochondrial function. Normal mitochondrial dynamics allow mitochondria to move to areas of increased metabolic demand, repair damaged mitochondria, maintain normal mitochondrial energetics, limit reactive oxygen species production, and isolate irreversibly damaged mitochondrial components for autophagy.¹⁸⁻²² The acute and chronic exposure to excessive nutrients, such as elevated glucose or free fatty acids, stimulates the mitochondrial network to undergo fission in multiple cells types, including human endothelial cells, leading to excessive mitochondrial ROS production.^{1,22-24} Seminal work in cultured endothelial cells demonstrates that high glucose driven excessive mitochondrial ROS production that leads both acute and chronic impairment endothelial cell function through cell signaling and epigenomic pathways.^{3,25} We have also previously demonstrated that reducing mitochondrial superoxide levels in resistance arteries from patients with type 2 diabetes reverses impaired endothelium-dependent vasodilation in human small resistance vessels.²

These data suggest that targeting mitochondrial dynamics proteins to reduce excessive mitochondrial fission could benefit vascular health in vessels acutely or chronically exposed to abnormal glucose levels. While there are multiple proteins involved in mitochondrial fusion and fission processes, Fis1, located on the outer mitochondrial membrane and docking protein for Drp1, has been repeatedly shown to be over-expressed in the setting of diabetes, acute high glucose exposure, or acute exposure to excessive free fatty acids in multiple cell types.^{1,26-29} While Fis1 is not necessary for all fission,³⁰ Fis1-Drp1 mediated fission appears to be preferred under conditions of cellular stress such as hypoxia and excessive glucose exposure.⁹⁻¹² Prior work in the human vasculature demonstrate Fis1 is overexpressed in endothelial cells from individuals with type 2 diabetes.¹ In addition, high glucose exposure leads to over-expression of Fis1 in cultured human aortic endothelial cells, and molecular silencing of Fis1 or Drp1 expression in human aortic endothelial cells exposed to high glucose results in an increase in phosphorylation of eNOS at its Ser1177 activation site.¹ Additionally, we previously showed that clinically relevant levels of low glucose exposure leads to mitochondrial fission in human endothelial cells and excessive mtROS production that can be reversed by suppressing mitochondrial fission protein activity.⁸ Our new data significantly extends the translational impact of prior work by showing that molecular inhibition of Fis1 reverses impaired endothelium-dependent vasodilation in intact human resistance arteries, increases nitric oxide bioavailability in these vessels, and protects endothelial cell layer integrity in the setting of abnormal glucose levels. In addition, we determined that targeting Fis1 does not impact endothelial cell mitochondrial metabolism or the expression of other mitochondrial proteins involved with dynamics or autophagy, reassuring observations when considering potential off-target effects of targeting the Fis1 as a therapeutic.

We found the pep213, designed to bind Fis1 at a key interaction surface, both increased NO bioavailability in cultured endothelial cells and reversed both type 2 diabetes and high glucose-induced impairment of endothelium-dependent vasodilation. Together with our molecular data, our data using the novel pep213 peptide support that concept that direct pharmacological inhibition of Fis1 has favorable effects on the diabetic vasculature. Interestingly, several medications given in the treatment of type 2 diabetes, two of which improve reduce cardiovascular risk in T2DM, also inhibit mitochondrial fission as an “off-target” effect by reducing Fis1 and/or Drp1 expression. Empagliflozin, and SGLT2 inhibitor with favorable effects on cardiovascular risk, mortality, and microvascular renal disease in T2DM patients,^{31,32} used in rat model of T2DM (OLEFT), reduced Fis1 over-expression in the model, reduced mitochondrial fission, and up-regulated mitochondrial superoxide dismutase, SOD2, in cardiac myocytes.³³ Metformin, a long-time first-line agent for T2DM glucose control with favorable cardiovascular effects, reduces atherosclerotic formation in ApoE knockout mice by suppressing Drp1-mediated fission.³⁴ The dipeptidyl peptidase 4 inhibitor vildagliptin, from a class of medications known to increase NO production, reduces expression of Fis1 and Drp1, reduces Drp1 translocation from the cytosol, reduces mitochondrial fission and ROS production, while increasing NO production in the aortic endothelium of diabetic mice.³⁵ Interpreted within the framework of our data, these common anti-diabetes medications may have ameliorative vascular effects in T2DM in part based on off target effects on the expression and/or interaction of Fis1 and Drp1. Whether these improvements are due to improved glycemic control or direct inhibition of Fis1 or Drp1 interaction merits future study.

Our study has some limitations. First, we focused primarily on the Fis1-Drp1 axis in regulating the vascular effects of T2DM and abnormal glucose exposures due to prior data supporting this axis as being most relevant in the setting of hyperglycemia and other pathological stimuli and our prior work supporting Fis1's pathophysiological role in the diabetic endothelium.^{1, 9-12} Whether or not blocking Fis1 impairs interactions with Drp1 or with other mitochondrial docking proteins (Mff, MiD49/51) is unknown and merits future investigation. We did also not focus on mitochondrial fusion proteins (e.g. OPA1, Mfn1, Mfn2). Emerging data suggest Mfn2 expression downregulated in the tissues of patients with T2DM and Fis1 may also drive fission by inhibiting the GTPase activity of fusion proteins which may further reduce NO bioavailability.^{1, 36-38} The fusion pathway's role in the regulation of human vascular endothelial function and its potential interaction with Fis1 merits also merits additional investigation. We did not perform osmotic controls with our experiments. However, we have done these controls with similar intact vessel and endothelial cell experiments on mitochondrial regulation of vascular function in our prior work and have never shown osmotic differences to influence our results.^{4,8}, Balanced against these limitations is the novelty of our findings in highly disease relevant tissues from humans, and the development of novel intervention to blunt and reverse the adverse effects of diabetes on the human vasculature targeting the mitochondrial fission machinery.

Supplemental Results:

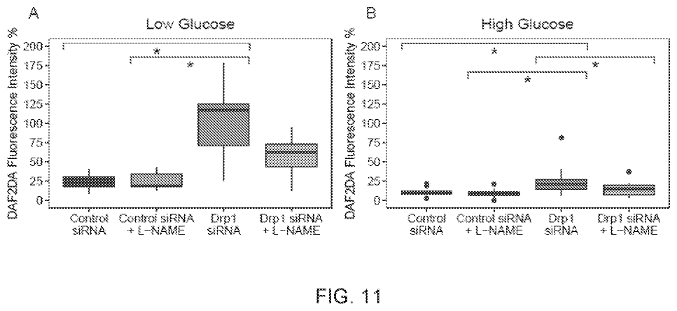

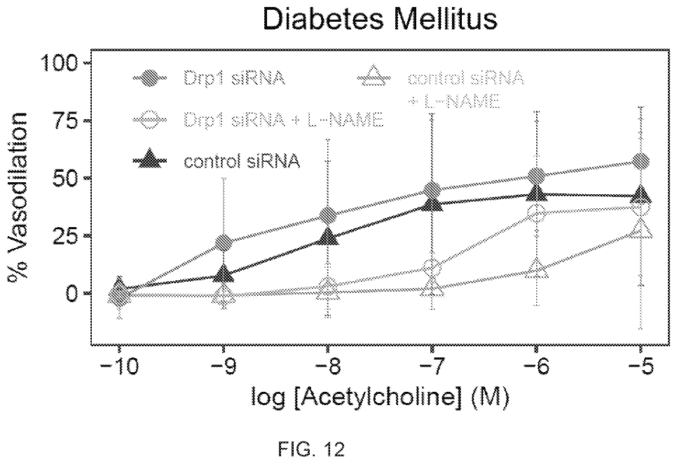

Consistent with excessive mitochondrial fission in impairing NO-dependent vasodilation in the diabetic endothelium, we previously reported similar findings upon genetic silencing of the mitochondrial fission mechanoenzyme Drp1.8 In those studies, siRNA to Drp1 significantly reduces Drp1 expression in human arterioles and prevented LG-induced impairment endothelium-dependent vasodilation in resistance arteries from healthy humans.8 In the current study, we found that genetic silencing of Drp1 also prevented HG-induced impairment of endothelium-dependent vasodilation, (FIG. 10, N=6, P<0.0001 overall vs. all other exposures), which was completely abolished by L-NAME but not papaverine (data not shown). Additionally, DAF2-DA fluorescence was also significantly increased in vessels from healthy individuals exposed to HG (FIG. 11A; n=9, P=0.02 overall P<0.05 for Drp1 siRNA vs. scrambled control, scrambled control+LNAME, and Drp1 siRNA+L-NAME) and LG (FIG. 11B, n=5, P=0.003 overall, P<0.04 Fis1 siRNA vs. all other exposures). L-NAME completely abolished the increases in DAF2-DA fluorescence in both cases. Additionally, transfection with Drp1 siRNA showed a trend toward improved endothelium-dependent vasodilation in vessels from humans with type 2 diabetes (FIG. 12).

### Conclusion:

The work presented here demonstrates the critical role of Fis1 in regulating vascular endothelial function in intact resistance vessels from individuals with T2DM and in settings of acute abnormal glucose levels. We used both molecular techniques as well as a novel peptide, pep213, specifically designed in to block a key binding surface of Fis1, to demonstrate the importance of this interaction to vascular function in vessels from T2DM patients. We additionally showed that reducing Fis1 levels does not impact the levels of other important mitochondrial proteins or mitochondrial oxygen consumption while improving endothelial function. Taken together with earlier works, these data support further work into the pharmacological targeting of Fis1 to reduce vascular complications in T2DM.

### Example 2

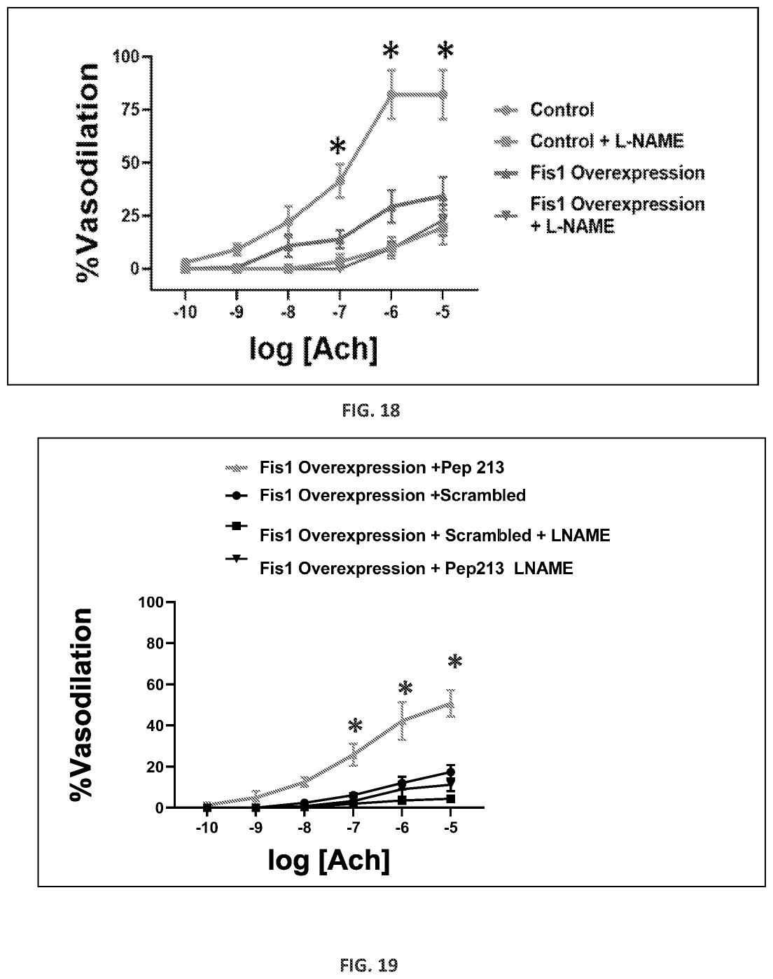

The inventors further demonstrated that overexpression of Fis1 in resistance arterioles from healthy human (transfected with a plasmid for endothelial-specific over-expression of human Fis1 with a 48 hour incubation period) results in impaired endothelium-dependent vasodilation in an eNOS-dependent manner (as determined by the loss in acetylcholine induced endothelium-dependent vasodilation with the use of eNOS inhibitor L-NAME). N=5, P<0.001 overall. *P<0.05 at the indicated doses of acetylcholine as demonstrated in FIG. 18. Further the peptide of the present invention, e.g., Pep213, can reverse impaired endothelium-dependent vasodilation in resistance arterioles. One hour of exposure to pep213 attached to a tat sequence to improve cell penetration (1 μM pep213-tat) reverses impaired endothelium-dependent vasodilation in resistance arterioles from healthy humans over-expressing Fis1 in the endothelium (overexpression of human Fis1 achieved using lentiviral vector to transfect the vessel with a plasmid for endothelial-specific over-expression of human Fis1). 1 μM of a scrambled peptide using the same amino acids as pep213 in a random order has no effect on endothelium dependent vasodilation to acetylcholine. Pep213-tat induce improves endothelium-dependent vasodilation in an eNOS-dependent manner (as determined by the loss in acetylcholine induced endothelium-dependent vasodilation with the use of eNOS inhibitor L-NAME). N=5, P<0.001 overall. *P<0.05 at the indicated doses of acetylcholine.

Further, co-crystalization of recombinant human Fis1 with Pep213 were performed. Peptides were then resuspended in Fis1 buffer. Crystals were grown by using hanging drop vapor diffusion and flash-frozen after rapid-transfer into a cryo-protectant solution followed by placement in liquid nitrogen. Multiple data sets were collected remotely at the Advanced Photon Source (Argonne, IL) LS-CAT beamline 21-ID-F with a MD2-S Microdiffractometer and Rayonix MX300 detector. A total of 180° of data were collected in 0.5° increments with a detector distance of 260 mm. All data were processed using XDS. Molecular replacement was performed with Phenix Phaser-MR followed by an auto-build step using Phenix AutoBuild. Refinement was performed using Phenix.Refine and WinCoot. Final structure resolution was 1.85 Å. The co-complex structure (A) clearly shows pep213 engaging with Fis1 via a variety of bonding interactions including salt-bridge formation, hydrogen bonding, and Van der Waals interactions.

The amino acids of the Pep213 important for Fis1 binding were determined by sequential replacement of each of the 14 amino acids with alanine, shown in Table 2. Microscale thermophoresis was used to determine critical pep213 residues for the Fis1-pep213 interaction. Each residue in pep213 was sequentially replaced with an alanine, for a total of 14 peptides. Peptides were then resuspended in Fis1 buffer. Microscale thermophoresis experiments were performed at 25° C. using a NanoTemper Monolith NT.115 instrument with a 16-point dilution series (1:1 dilution) of each peptide against a fixed concentration of fluorescently labelled Fis1. Data analysis were performed using Nanotemper MO. Affinity Analysis software to determine K_{D} values. Binding affinity values were used to determine the ΔG° of the reaction (ΔG°-RTlnK) which was then used to calculate a ΔΔG°⁰ value (ΔG°ₚₑₚ₂₁₃-ΔG°ᵥₐᵣᵢₐₙₜ). A subset of residues on each terminus of the peptide do not contribute significantly to binding, as indicated by a lower ΔΔG° value (B).

TABLE 2
Sequences and mutational analyzed Pep213 peptides

| Sequence ID/Name | Sequence | Comments |
|---|---|---|
| Pep213 (SEQ ID NO: 1) | SHKHDPLPYPHFLL | Fis1 inhibitory peptide |
| | | (pep213) |
| TAT (SEQ ID NO: 2) | YGRKKRRQRRR | Cell penetrating peptide |
| Pep213 fused to TAT | YGRKKRRQRRRGSGSGSSHKH | Pep213-linker-TAT |
| (SEQ ID NO: 3) | DPLPYPHFLL | |
| Linker (SEQ ID NO: 4) | GSGSGS | |
| siRNA to Fis1 (SEQ ID | rGrGrUrGrCrGrGrArGrCrArArGr | |
| NO: 5) | UrArCrArArUrGrArUrGAC | |
| siRNA to Fis1 (SEQ ID | rArCrUrArCrCrGrGrCrUrCrArAr | |
| NO: 6) | GrGrArArUrArCrGrArGAA | |
| siRNA to Fis1 (SEQ ID | rArCrArGrUrArGrArCrUrGrUrAr | |
| NO: 7) | GrUrGrUrGrArGrGrCrUCG | |
| siRNA to Drp1 (SEQ ID | rArGrArGrUrGrUrArArCrUrGrAr | |
| NO: 8 | UrUrCrArArUrCrCrGrUGA | |
| sirRNA to Drp1 (SEQ | rArGrGrArUrArUrUrGrArGrCrUr | |
| ID NO: 9) | UrCrArArArUrCrArGrAGA | |
| siRNA to Drp1 (SEQ ID | rCrCrCrUrUrArArArCrUrGrArGr | |
| NO: 10 | UrCrArArGrArUrCrUrGAA | |
| SEQ ID NO: 11-15 | SGSG, GSGS, SSSS, GGGS, | |
| (Linkers) | GGAAY | |
| Mutant Pep213 (SEQ ID | AHKHDPLPYPHFLL | Position 1 replaced with A |
| NO: 16) | | |
| Mutant Pep213 (SEQ ID | SAKHDPLPYPHFLL | Position 2 replaced with A |
| NO: 17) | | |
| Mutant Pep213 (SEQ ID | SHAHDPLPYPHFLL | Position 3 replaced with A |
| NO: 18) | | |
| Mutant Pep213 (SEQ ID | SHKADPLPYPHFLL | Position 4 replaced with A |
| NO: 19) | | |
| Mutant Pep213 (SEQ ID | SHKHAPLPYPHFLL | Position 5 replaced with A |
| NO: 20) | | |
| Mutant Pep213 (SEQ ID | SHKHDALPYPHFLL | Position 6 replaced with A |
| NO: 21) | | |
| Mutant Pep213 (SEQ ID | SHKHDPAPYPHFLL | Position 7 replaced with A |
| NO: 22) | | |
| Mutant Pep213 (SEQ ID | SHKHDPLAYPHFLL | Position 8 replaced with A |
| NO: 23) | | |
| Mutant Pep213 (SEQ ID | SHKHDPLPAPHFLL | Position 9 replaced with A |
| NO: 24) | | |
| Mutant Pep213 (SEQ ID | SHKHDPLPYAHFLL | Position 10 replaced with |
| NO: 25) | | A |
| Mutant Pep213 (SEQ ID | SHKHDPLPYPAFLL | Position 11 replaced with |
| NO: 26) | | A |
| Mutant Pep213 (SEQ ID | SHKHDPLPYPHALL | Position 12 replaced with |
| NO: 27) | | A |
| Mutant Pep213 (SEQ ID | SHKHDPLPYPHFAL | Position 13 replaced with |
| NO: 28) | | A |
| Mutant Pep213 (SEQ ID | SHKHDPLPYPHFLA | Position 14 replaced with |
| NO: 29) | | A |
| Mutant Pep213 (SEQ ID | XXXXXXLPYPXFLX | Based on FIG. 20, X can be |
| NO: 30) | | any amino acid, A or |
| | | residue corresponding to |
| | | SEQ ID NO: 1 |
| Pep213-linker-Tat | YGRKKRRQRRRXSHKHDPLPYP | Wherein X is an amino |
| (SEQ ID NO: 31) | HFLL | acid linker of 4-8 amino |
| | | acids, preferably G and S |
| mPep213-linker | YGRKKRRQRRRZXXXXXXLPY | X, each X can be A or the |
| (Y)-TAT | PXFLX | amino acid residue from |
| (SEQ ID NO: 32) | | SEQ ID NO: 1 and Z is a |
| | | 4-10 amino acid linker, |
| | | preferably of G and S |
| corePep213 (SEQ ID | LPYPX | Wherein X is any amino |
| NO: 33) | | acid, A or H. |
| corePep213-2 (SEQ ID | LPYPXF | Wherein X is any amino |
| NO: 34) | | acid, A or H. |
| corePep213-3 (SEQ ID | LPYPHFL | |
| NO: 35) | | |
| Core Pep213-4 (SEQ ID | LPYPHFLL | |
| NO: 36) | | |
| CorePep213-5 (SEQ ID | XLPYPHFL | Wherein X is from 1-30 |
| NO: 37) | | amino acids of any |
| | | sequence |
| corePep213-6 (SEQ ID | XLPYPHZ | Wherein X and Z are from |
| NO: 38) | | 0-30 amino acids of any |
| | | sequence (e.g. A or G) |
| corePep213-linker-TAT | X₁LPYPHZ-X₂-YGRKKRRQRRR | X₁ and Z are from 0-30 |
| (SEQ ID NO: 39) | | amino acid peptide (made |
| | | of any amino acids, e.g. A |
| | | or G, etc). |

Supplemental TABLE 1
Demographic, clinical, and medication information and in vivo vascular function
for 16 subjects with resistance arterioles that underwent Fis1 siRNA transfection
and studied for vasodilation in response to acetylcholine

| | Non-Diabetic | Non-Diabetic | Type 2 DM | |
|---|---|---|---|---|
| | (LG; n = 6) | (HG; n = 5) | (n = 6) | p-value |
| Age | 35 ± 17 | 58 ± 5 | 61 ± 10 | 0.01 |
| Sex (#female) | 3 | 3 | 0 | 0.12 |
| Smoking Status (#never) | 1 | 1 | 3 | 0.03 |
| History of Hypertension | 0 | 0 | 5 | 0.0002 |
| History of High Cholesterol | 0 | 0 | 5 | |
| Body mass Index (kg/m2) | 28 ± 5 | 26 ± 3 | 29 ± 5 | 0.80 |
| Waist Circumference (cm) | 95 ± 18 | 88 ± 10 | 109 ± 4 | 0.18 |
| Systolic Blood Pressure (mmHg) | 120 ± 17 | 126 ± 11 | 126 ± 11 | 0.69 |
| Diastolic Blood Pressure | 72 ± 10 | 74 ± 11 | 74 ± 8 | 0.85 |
| (mmHg) | | | | |
| Fasting Glucose (mg/dL) | 83 ± 11 | 82 ± 7 | 174 ± 35 | <0.0001 |
| Hemoglobin A1C (%) | 5.3 ± 0.3 | 5.4 ± 0.2 | 8.3 ± 1.8 | 0.0009 |
| Creatinine (mg/dL) | 0.82 ± 0.16 | 0.92 ± 0.18 | 0.95 ± 0.19 | 0.25 |
| Total Cholesterol (mg/dL) | 204 ± 33 | 195 ± 16 | 174 ± 41 | 0.35 |
| HDL Cholesterol | 71 ± 15 | 86 ± 19 | 49 ± 6 | 0.009 |
| LDL Cholesterol (mg/dL) | 115 ± 34 | 95 ± 18 | 84 ± 31 | 0.21 |

Medications (% on Therapy)

| | | | |
|---|---|---|---|
| Biguanide | 0 | 0 | 83 |
| Sulfonylurea | 0 | 0 | 83 |
| Thiazoladinedione | 0 | 0 | 0 |
| DPP4 inhibitor | 0 | 0 | 33 |
| GLP-1 Agonist | 0 | 0 | 0 |
| Insulin | 0 | 0 | 0 |
| HMG CoA reductase Inhibitor | 0 | 0 | 100 |
| ACE Inhibitor | 0 | 0 | 83 |
| Angiotensin II Receptor Blocker | 0 | 0 | 17 |
| GLP1 Agonist | 0 | 0 | 17 |
| SGLT2 Inhibitor | 0 | 0 | 0 |

DM—Diabetes

Supplemental TABLE 2
Demographic, clinical, and medication information and in
vivo vascular function for 17 subjects with resistance arterioles
that underwent Fis1 siRNA transfection and studied for biological
NO production using DAF2DA fluorescence.

| | Non-DM | Non-DM | p- |
|---|---|---|---|
| | (LG; n = 8) | (HG; n = 9) | value |
| Age | 42 ± 16 | 35 ± 14 | 0.54 |
| Sex (#female) | 5 | 8 | 0.35 |
| Smoking Status (#never) | 3 | 2 | 0.38 |
| History of Hypertension | 0 | 0 | — |
| History of High Cholesterol | 0 | 0 | — |
| Body mass Index (kg/m2) | 23 ± 3 | 23 ± 6 | 0.89 |
| Waist Circumference (cm) | 85 ± 11 | 82 ± 17 | 0.78 |
| Systolic Blood Pressure (mmHg) | 125 ± 9 | 111 ± 12 | 0.02 |
| Diastolic Blood Pressure (mmHg) | 68 ± 5 | 68 ± 12 | 0.82 |
| Fasting Glucose (mg/dL) | 85 ± 3 | 89 ± 9 | 0.50 |
| Hemoglobin A1C (%) | 5 ± 0.2 | 5.2 ± 0.3 | 0.27 |
| Creatinine (mg/dL) | 0.8 ± 0.1 | 0.74 ± 0.11 | 0.12 |
| Total Cholesterol (mg/dL) | 197 ± 31 | 171 ± 22 | 0.14 |
| HDL Cholesterol | 70 ± 16 | 63 ± 23 | 0.81 |
| LDL Cholesterol (mg/dL) | 109 ± 23 | 89 ± 22 | 0.12 |

DM—Diabetes

Supplemental TABLE 3
Demographic, clinical, and medication information and in
vivo vascular function for 13 subjects with resistance
arterioles that underwent Drp1 siRNA transfection and
studied for vasodilation in response to acetylcholine.

| | Non-Diabetic | Type 2 DM | |
|---|---|---|---|
| | (HG; n = 5) | (n = 4) | p-value |
| Age | 33 ± 11 | 46 ± 15 | 0.43 |
| Sex (#female) | 2 | 3 | 0.29 |
| Smoking Status (#never) | 5 | 2 | 0.49 |
| History of Hypertension | 0 | 3 | 0.01 |
| History of High Cholesterol | 0 | 2 | 0.20 |
| Body mass Index (kg/m2) | 27 ± 7 | 30 ± 5 | 0.49 |
| Waist Circumference (cm) | 89 ± 19 | 95 ± 12 | 0.75 |
| Systolic Blood Pressure | 119 ± 20 | 131 ± 33 | 0.96 |
| (mmHg) | | | |
| Diastolic Blood Pressure | 74 ± 15 | 79 ± 22 | 0.95 |
| (mmHg) | | | |
| Fasting Glucose (mg/dL) | 71 ± 9 | 142 ± 55 | 0.002 |
| Hemoglobin A1C (%) | 5.2 ± 0.5 | 8.4 ± 1.0 | <0.0001 |
| Creatinine (mg/dL) | 0.89 ± 0.23 | 0.82 ± 0.22 | 0.40 |
| Total Cholesterol (mg/dL) | 179 ± 18 | 163 ± 16 | 0.09 |
| HDL Cholesterol | 74 ± 27 | 56 ± 3 | 0.39 |
| LDL Cholesterol (mg/dL) | 87 ± 13 | 90 ± 19 | 0.40 |

Medications (% on Therapy)

| | | |
|---|---|---|
| Biguanide | 0 | 75 |
| Sulfonylurea | 0 | 25 |
| Thiazoladinedione | 0 | 0 |
| DPP4 inhibitor | 0 | 0 |
| GLP-1 Agonist | 0 | 0 |
| Insulin | 0 | 25 |
| HMG CoA reductase Inhibitor | 0 | 50 |
| ACE Inhibitor | 0 | 25 |
| Angiotensin II Receptor Blocker | 0 | 0 |
| SGLT2 Inhibitor | 0 | 0 |

DM-Diabetes

Supplemental TABLE 4
Demographic, clinical, and medication information and in
vivo vascular function for 16 subjects with resistance arterioles
that underwent Drp1 siRNA transfection and studied for biological
NO production using DAF2DA fluorescence.

| | Non-Diabetic | Type 2 DM | p- |
|---|---|---|---|
| | (LG; n = 5) | (HG; n = 9) | value |
| Age | 29 ± 9 | 42 ± 14 | 0.27 |
| Sex (#female) | 3 | 6 | 0.37 |
| Smoking Status (#never) | 3 | 2 | 0.36 |
| History of Hypertension | 0 | 0 | na |
| History of High Cholesterol | 0 | 0 | na |
| Body mass Index (kg/m2) | 25 ± 6 | 29 ± 6 | 0.26 |
| Waist Circumference (cm) | 86 ± 16 | 94 ± 15 | 0.70 |
| Systolic Blood Pressure | 116 ± 10 | 126 ± 12 | 0.61 |
| (mmHg) | | | |
| Diastolic Blood Pressure | 71 ± 11 | 71 ± 10 | 0.91 |
| (mmHg) | | | |
| Fasting Glucose (mg/dL) | 82 ± 13 | 87 ± 9 | 0.45 |
| Hemoglobin A1C (%) | 5.1 ± 0.1 | 5.2 ± 0.3 | 0.42 |
| Creatinine (mg/dL) | 0.81 ± 0.15 | 0.86 ± 0.21 | 0.53 |
| Total Cholesterol (mg/dL) | 170 ± 35 | 178 ± 27 | 0.72 |
| HDL Cholesterol | 67 ± 10 | 61 ± 12 | 0.74 |
| LDL Cholesterol (mg/dL) | 86 ± 29 | 97 ± 20 | 0.76 |

DM—Diabetes

Supplemental TABLE 5
Demographic, clinical, and medication information and
in vivo vascular function for 10 subjects with resistance
arterioles exposed to pep213 and studied for vasodilation
in response to acetylcholine in FIG. 7

| | Non-DM | T2DM | p- |
|---|---|---|---|
| | (HG; n = 6) | (n = 4) | value |
| Age | 35 ± 14 | 46 ± 15 | 0.02 |
| Sex (#female) | 1 | 2 | 0.29 |
| Smoking Status (#never) | 5 | 1 | 0.49 |
| History of Hypertension | 0 | 3 | 0.01 |
| History of High Cholesterol | 0 | 3 | 0.20 |
| Body mass Index (kg/m2) | 27 ± 7 | 30 ± 5 | 0.007 |
| Waist Circumference (cm) | 89 ± 19 | 95 ± 12 | 0.04 |
| Systolic Blood Pressure | 117 ± 7 | 125 ± 16 | 0.32 |
| (mmHg) | | | |
| Diastolic Blood Pressure | 70 ± 5 | 77 ± 7 | 0.10 |
| (mmHg) | | | |
| Fasting Glucose (mg/dL) | 92 ± 6 | 129 ± 36 | 0.04 |
| Hemoglobin A1C (%) | 5.0 ± 0.3 | 8.0 ± 2.4 | 0.01 |
| Creatinine (mg/dL) | 0.92 ± 0.16 | 0.77 ± 0.07 | 0.11 |
| Total Cholesterol (mg/dL) | 179 ± 35 | 179 ± 55 | 0.98 |
| HDL Cholesterol | 55 ± 19 | 50 ± 18 | 0.67 |
| LDL Cholesterol (mg/dL) | 110 ± 22 | 104 ± 41 | 0.79 |

Medications (% on Therapy)

| | | |
|---|---|---|
| Biguanide | 0 | 100 |
| Sulfonylurea | 0 | 50 |
| Thiazoladinedione | 0 | 0 |
| GLP-1 Agonist | 0 | 25 |
| DPP4 inhibitor | 0 | 0 |
| Insulin | 0 | 25 |
| HMG CoA reductase Inhibitor | 0 | 50 |
| ACE Inhibitor | 0 | 25 |
| Angiotensin II Receptor Blocker | 0 | 25 |
| SGLT2 Inhibitor | 0 | 0 |

DM—Diabetes

Supplemental TABLE 6
Demographic, clinical, and medication information and in vivo
vascular function for 9 subjects with resistance arterioles
exposed to pep213-tat and scrambled peptide-tat and studied
for vasodilation in response to acetylcholine in FIG. 8

| | Non-DM | T2DM | p- |
|---|---|---|---|
| | (HG; n = 5) | (n = 4) | value |
| Age | 54 ± 19 | 46 ± 15 | 0.41 |
| Sex (#female) | 4 | 2 | 0.2887 |
| Smoking Status (#smoker) | 0 | 0 | — |
| History of Hypertension | 1 | 1 | 0.72 |
| History of High Cholesterol | 0 | 1 | 0.56 |
| Body mass Index (kg/m2) | 29 ± 6 | 35 ± 10 | 0.27 |

Medications (% on Therapy)

| | | | |
|---|---|---|---|
| ACE Inhibitor | 0 | 0 | — |
| Angiotensin II Receptor Blocker | 0 | 1 | 0.56 |

DM—Diabetes

### REFERENCES

1. Shenouda S M, Widlansky M E, Chen K, Xu G, Holbrook M, Tabit C E, Hamburg N M, Frame A A, Caiano T L, Kluge M A, Duess M A, Levit A, Kim B, Hartman M L, Joseph L, Shirihai O S and Vita J A. Altered mitochondrial dynamics contributes to endothelial dysfunction in diabetes mellitus. Circulation. 2011; 124:444-453.
2. Kizhakekuttu T J, Wang J, Dharmashankar K, Ying R, Gutterman D D, Vita J A and Widlansky M E. Adverse alterations in mitochondrial function contribute to type 2 diabetes mellitus-related endothelial dysfunction in humans. Arterioscler Thromb Vasc Biol. 2012; 32:2531-2539.
3. El-Osta A, Brasacchio D, Yao D, Pocai A, Jones P L, Roeder R G, Cooper M E and Brownlee M. Transient high glucose causes persistent epigenetic changes and altered gene expression during subsequent normoglycemia. J Exp Med. 2008; 205:2409-2417.
4. Wang J, Alexanian A, Ying R, Kizhakekuttu T J, Dharmashankar K, Vasquez-Vivar J, Gutterman D D and Widlansky M E. Acute Exposure to Low Glucose Rapidly Induces Endothelial Dysfunction and Mitochondrial Oxidative Stress: Role for AMP Kinase. Arterioscler Thromb Vasc Biol. 2012; 32:712-720.
5. Yusuf S, Dagenais G, Pogue J, Bosch J and Sleight P. Vitamin E supplementation and cardiovascular events in high-risk patients. The Heart Outcomes Prevention Evaluation Study Investigators. N Engl J Med. 2000; 342:154-160.
6. Willcox B J, Curb J D and Rodriguez B L. Antioxidants in cardiovascular health and disease: key lessons from epidemiologic studies. Am J Cardiol. 2008; 101:75D-86D.
7. Widlansky M E and Hill R B. Mitochondrial regulation of diabetic vascular disease: an emerging opportunity. Transl Res. 2018.
8. Tanner M J, Wang J, Ying R, Suboc T B, Malik M, Couillard A, Branum A, Puppala V and Widlansky M E. Dynamin-related protein 1 mediates low glucose-induced endothelial dysfunction in human arterioles. Am J Physiol Heart Circ Physiol. 2017; 312:H515-H527.
9. Kim H, Scimia M C, Wilkinson D, Trelles R D, Wood M R, Bowtell D, Dillin A, Mercola M and Ronai Z A. Fine-tuning of Drp1/Fis1 availability by AKAP121/Siah2 regulates mitochondrial adaptation to hypoxia. Mol Cell. 2011; 44:532-544.
10. Kumari S, Anderson L, Farmer S, Mehta S L and Li P A. Hyperglycemia alters mitochondrial fission and fusion proteins in mice subjected to cerebral ischemia and reperfusion. Transl Stroke Res. 2012; 3:296-304.
11. Ciarlo L, Manganelli V, Garofalo T, Matarrese P, Tinari A, Misasi R, Malorni W and Sorice M. Association of fission proteins with mitochondrial raft-like domains. Cell Death Differ. 2010; 17:1047-1058.
12. Kaddour-Djebbar I, Choudhary V, Brooks C, Ghazaly T, Lakshmikanthan V, Dong Z and Kumar M V. Specific mitochondrial calcium overload induces mitochondrial fission in prostate cancer cells. Int J Oncol. 2010; 36:1437-1444.
13. Widlansky M E, Jensen D M, Wang J, Liu Y, Geurts A M, Kriegel A J, Liu P, Ying R, Zhang G, Casati M, Chu C, Malik M, Branum A, Tanner M J, Tyagi S, Usa K and Liang M. miR-29 contributes to normal endothelial function and can restore it in cardiometabolic disorders. EMBO Mol Med. 2018.
14. American Diabetes A. 2. Classification and Diagnosis of Diabetes: Standards of Medical Care in Diabetes-2019. Diabetes Care. 2019; 42:S13-S28.
15. Suboc™, Dharmashankar K, Wang J, Ying R, Couillard A, Tanner M J and Widlansky M E. Moderate Obesity and Endothelial Dysfunction in Humans: Influence of Gender and Systemic Inflammation. Physiol Rep. 2013; 1.
16. Mohandas A, Suboc T B, Wang J, Ying R, Tarima S, Dharmashankar K, Malik M and Widlansky M E. Mineralocorticoid exposure and receptor activity modulate microvascular endothelial function in African Americans with and without hypertension. Vasc Med. 2015.
17. Serasinghe M N, Seneviratne A M, Smrcka A V and Yoon Y. Identification and characterization of unique proline-rich peptides binding to the mitochondrial fission protein hFis1. J Biol Chem. 2010; 285:620-30.
18. Chen H and Chan D C. Mitochondrial dynamics-fusion, fission, movement, and mitophagy-in neurodegenerative diseases. Hum Mol Genet. 2009; 18:R169-R176.
19. Chan D C. Fusion and fission: interlinked processes critical for mitochondrial health. Annu Rev Genet. 2012; 46:265-287.
20. Detmer S A and Chan D C. Functions and dysfunctions of mitochondrial dynamics. Nat Rev Mol Cell Biol. 2007; 8:870-9.
21. Suen D F, Norris K L and Youle R J. Mitochondrial dynamics and apoptosis. Genes Dev. 2008; 22:1577-90.
22. Yu T, Robotham J L and Yoon Y. Increased production of reactive oxygen species in hyperglycemic conditions requires dynamic change of mitochondrial morphology. Proc Natl Acad Sci USA. 2006; 103:2653-2658.
23. Molina A J, Wikstrom J D, Stiles L, Las G, Mohamed H, Elorza A, Walzer G, Twig G, Katz S, Corkey B E and Shirihai O S. Mitochondrial networking protects beta-cells from nutrient-induced apoptosis. Diabetes. 2009; 58:2303-15.
24. Jheng H F, Tsai P J, Guo S M, Kuo L H, Chang C S, Su I J, Chang C R and Tsai Y S. Mitochondrial fission contributes to mitochondrial dysfunction and insulin resistance in skeletal muscle. Mol Cell Biol. 2012; 32:309-19.
25. Nishikawa T, Edelstein D, Du X L, Yamagishi S, Matsumura T, Kaneda Y, Yorek M A, Beebe D, Oates P J, Hammes H P, Giardino I and Brownlee M. Normalizing mitochondrial superoxide production blocks three pathways of hyperglycaemic damage. Nature. 2000; 404:787-790.
26. Liu N, Wu J, Zhang L, Gao Z, Sun Y, Yu M, Zhao Y, Dong S, Lu F and Zhang W. Hydrogen Sulphide modulating mitochondrial morphology to promote mitophagy in endothelial cells under high-glucose and high-palmitate. J Cell Mol Med. 2017; 21:3190-3203.
27. Gioscia-Ryan R A, Battson M L, Cuevas L M, Zigler M C, Sindler A L and Seals D R. Voluntary aerobic exercise increases arterial resilience and mitochondrial health with aging in mice. Aging (Albany NY). 2016; 8:2897-2914.
28. Choi H Y, Park J H, Jang W B, Ji S T, Jung S Y, Kim da Y, Kang S, Kim Y J, Yun J, Kim J H, Baek S H and Kwon S M. High Glucose Causes Human Cardiac Progenitor Cell Dysfunction by Promoting Mitochondrial Fission: Role of a GLUT1 Blocker. Biomol Ther (Seoul). 2016; 24:363-70.
29. Zhang Y, Feng J, Wang Q, Zhao S, Yang S, Tian L, Meng P, Li J and Li H. Hyperglycaemia Stress-Induced Renal Injury is Caused by Extensive Mitochondrial Fragmentation, Attenuated MKP1 Signalling, and Activated JNK-CaMKII-Fis1 Biological Axis. Cell Physiol Biochem. 2018; 51:1778-1798.
30. Loson O C, Song Z, Chen H and Chan D C. Fis1, Mff, MiD49, and MiD51 mediate Drp1 recruitment in mitochondrial fission. Mol Biol Cell. 2013; 24:659-67.
31. Zinman B, Wanner C, Lachin J M, Fitchett D, Bluhmki E, Hantel S, Mattheus M, Devins T, Johansen O E, Woerle H J, Broedl U C, Inzucchi S E and Investigators E-RO. Empagliflozin, Cardiovascular Outcomes, and Mortality in Type 2 Diabetes. N Engl J Med. 2015; 373:2117-28.
32. Wanner C, Inzucchi S E, Lachin J M, Fitchett D, von Eynatten M, Mattheus M, Johansen O E, Woerle H J, Broedl U C, Zinman B and Investigators E-RO. Empagliflozin and Progression of Kidney Disease in Type 2 Diabetes. N Engl J Med. 2016; 375:323-34.
33. Mizuno M, Kuno A, Yano T, Miki T, Oshima H, Sato T, Nakata K, Kimura Y, Tanno M and Miura T. Empagliflozin normalizes the size and number of mitochondria and prevents reduction in mitochondrial size after myocardial infarction in diabetic hearts. Physiol Rep. 2018; 6:e13741.
34. Wang Q, Zhang M, Torres G, Wu S, Ouyang C, Xie Z and Zou M H. Metformin Suppresses Diabetes-Accelerated Atherosclerosis via the Inhibition of Drp1-Mediated Mitochondrial Fission. Diabetes. 2017; 66:193-205.
35. Liu H, Xiang H, Zhao S, Sang H, Lv F, Chen R, Shu Z, Chen A F, Chen S and Lu H. Vildagliptin improves high glucose-induced endothelial mitochondrial dysfunction via inhibiting mitochondrial fission. J Cell Mol Med. 2019; 23:798-810.
36. Yu R, Jin S B, Lendahl U, Nister M and Zhao J. Human Fis1 regulates mitochondrial dynamics through inhibition of the fusion machinery. EMBO J. 2019; 38.
37. Bach D, Pich S, Soriano F X, Vega N, Baumgartner B, Oriola J, Daugaard J R, Lloberas J, Camps M, Zierath J R, Rabasa-Lhoret R, Wallberg-Henriksson H, Laville M, Palacin M, Vidal H, Rivera F, Brand M and Zorzano A. Mitofusin-2 determines mitochondrial network architecture and mitochondrial metabolism. A novel regulatory mechanism altered in obesity. J Biol Chem. 2003; 278:17190-7.
38. Lugus J J, Ngoh G A, Bachschmid M M and Walsh K. Mitofusins are required for angiogenic function and modulate different signaling pathways in cultured endothelial cells. J Mol Cell Cardiol. 2011; 51:885-893.

We claim:

1. An inhibitory peptide of mitochondrial fission protein 1 (Fis1) activity comprising (a) an amino acid sequence of SEQ ID NO:38 (XLPYPHZ) or a sequence having at least 80% sequence identity to SEQ ID NO:38, wherein X and Z can be a peptide from 0-30 amino acids, optionally from 1-20 amino acids.

2. The inhibitory peptide of claim 1 comprising (a) an amino acid sequence selected from SEQ ID NO:33-37 or a sequence laving at least 80% sequence identity to SEQ ID NO:33-37, wherein the peptide is from about 5-50 amino acids in length, optionally 5-30 amino acids in length.

3. The inhibitory peptide of claim 1, wherein the amino acid sequence comprises (a) SEQ ID NO:1 (SHKHDPLPYPHFLL) or a sequence having at least 90% sequence identity to SEQ ID NO:1, or any one of SEQ ID NO:16-21, 26 and 29 or a sequence having 90% similarity to SEQ ID NO:16-21, 26 and 29.

4. The inhibitory peptide of claim 1, the inhibitory peptide comprising (a) linked to (b) a carrier or an amino acid sequence encoding a carrier peptide, a tag peptide, or cell binding peptide.

5. The inhibitory peptide of claim 4, wherein the inhibitory peptide comprises a carrier peptide.

6. The inhibitory peptide of claim 5, wherein the carrier peptide is a cell penetrating peptide sequence, optionally TAT (SEQ ID NO:2) or a sequence having at least 90% sequence identity to SEQ ID NO:2.

7. The inhibitory peptide of claim 4, wherein (a) and (b) are both peptides and are linked by a linker sequence.

8. The inhibitory peptide of claim 7, wherein the linker sequence is SEQ ID NO:4, 11, 12, 13, 14, or 15.

9. The inhibitory peptide of claim 1, wherein the peptide comprises SEQ ID NO:3
(YGRKKRRQRRRGSGSGSSHKHDPLPYPHFLL), SEQ ID NO:31, SEQ ID NO:32, SEQ ID NO: 39 or a peptide having at least 90% sequence identity to SEQ ID NO:3, SEQ ID NO:31, SEQ ID NO:32 or SEQ ID NO:39.

10. A polynucleotide encoding a Fis1 inhibitory peptide, the polynucleotide comprising heterologous promoter sequence and a polynucleotide sequence encoding the peptide of claim 1.

11. The polynucleotide of claim 10, wherein the polynucleotide is a vector.

12. A vector capable of expressing the inhibitory peptide, the vector comprising a promoter operably connected to a polynucleotide encoding the peptide of claim 1.

13. The vector of claim 12, further comprising heterologous backbone sequence.

14. A host cell comprising the vector of claim 12 and capable of expressing the inhibitory peptide.

15. A method of
(a) treating vascular complications associated with type 2 diabetes in a subject in need thereof;
(b) reversing impaired vasodilation in a subject in need thereof;
(c) increasing NO bioavailability in human microvascular endothelial cells in a subject in need thereof; or
(d) treating impaired endothelial function in a subject in need thereof;
the method comprising administering an effective amount of the Fis1 inhibitory peptide of claim 1.

16. (canceled)

17. The method of claim 15, wherein the subject has one or more of the following diseases or conditions: type 2 diabetes, high glucose-induced and type 2 diabetes-associated impairment of endothelium-dependent vasodilation in human resistance arteries in a nitric oxide synthase-dependent manner, atherosclerosis, cerebrovascular arterial disease, coronary arterial disease, renovascular disease, and peripherial arterial disease.

18.-23. (canceled)

24. A kit comprising one or more of the following:
(a) the peptide of claim 1;
(b) a polynucleotide encoding the peptide of claim 1;
(c) an expression vector comprising the polynucleotide of (b), wherein the polynucleotide of (b) is operably linked to a promoter for expression of the peptide of the encoded peptide; or
(d) a cell comprising the expression vector of (c);
and instructions for use.

25. A composition comprising the inhibitory peptide of any claim 1 and a pharmaceutically acceptable carrier.


SEQUENCE LISTING (39 sequences)

SEQ ID NO: 1 - protein, 14 aa, Artificial Sequence
  Synthetic - pep213, a Fis1 inhibitory peptide
SHKHDPLPYPHFLL

SEQ ID NO: 2 - protein, 11 aa, Artificial Sequence
  Synthetic - TAT, a cell penetrating peptide
YGRKKRRQRRR

SEQ ID NO: 3 - protein, 31 aa, Artificial Sequence
  Synthetic - Pep213 fused to TAT (pep213-TAT)
YGRKKRRQRRRGSGSGSSHKHDPLPYPHFLL

SEQ ID NO: 4 - protein, 6 aa, Artificial Sequence
  Synthetic - linker peptide
GSGSGS

SEQ ID NO: 5 - RNA, 48 nt, Artificial Sequence
  Synthetic - siRNA to Fis1
rgrgrurgrcrgrgrargrcrarargrurarcrararurgrarurgac

SEQ ID NO: 6 - RNA, 48 nt, Artificial Sequence
  Synthetic - siRNA to Fis1
rarcrurarcrcrgrgrcrurcrarargrgrararurarcrgrargaa

SEQ ID NO: 7 - RNA, 48 nt, Artificial Sequence
  Synthetic - siRNA to Fis1
rarcrargrurargrarcrurgrurargrurgrurgrargrgrcrucg

SEQ ID NO: 8 - RNA, 48 nt, Artificial Sequence
  Synthetic - siRNA to Drp1
rargrargrurgrurararcrurgrarururcrararurcrcrgruga

SEQ ID NO: 9 - RNA, 48 nt, Artificial Sequence
  Synthetic - siRNA to Drp1
rargrgrarurarururgrargrcrururcrarararurcrargraga

SEQ ID NO: 10 - RNA, 48 nt, Artificial Sequence
  Synthetic - siRNA to Drp1
rcrcrcrururarararcrurgrargrurcrarargrarurcrurgaa

SEQ ID NO: 11 - protein, 4 aa, Artificial Sequence
  Synthetic - linker peptide
SGSG

SEQ ID NO: 12 - protein, 4 aa, Artificial Sequence
  Synthetic - linker peptide
GSGS

SEQ ID NO: 13 - protein, 4 aa, Artificial Sequence
  Synthetic - linker peptide
SSSS

SEQ ID NO: 14 - protein, 4 aa, Artificial Sequence
  Synthetic - linker peptide
GGGS

SEQ ID NO: 15 - protein, 5 aa, Artificial Sequence
  Synthetic - linker peptide
GGAAY

SEQ ID NO: 16 - protein, 14 aa, Artificial Sequence
  Synthetic - mutant pep213
AHKHDPLPYPHFLL

SEQ ID NO: 17 - protein, 14 aa, Artificial Sequence
  Synthetic - mutant pep213
SAKHDPLPYPHFLL

SEQ ID NO: 18 - protein, 14 aa, Artificial Sequence
  Synthetic - mutant pep213
SHAHDPLPYPHFLL

SEQ ID NO: 19 - protein, 14 aa, Artificial Sequence
  Synthetic - mutant pep213
SHKADPLPYPHFLL

SEQ ID NO: 20 - protein, 14 aa, Artificial Sequence
  Synthetic - mutant pep213
SHKHAPLPYPHFLL

SEQ ID NO: 21 - protein, 14 aa, Artificial Sequence
  Synthetic - mutant pep213
SHKHDALPYPHFLL

SEQ ID NO: 22 - protein, 14 aa, Artificial Sequence
  Synthetic - mutant pep213
SHKHDPAPYPHFLL

SEQ ID NO: 23 - protein, 14 aa, Artificial Sequence
  Synthetic - mutant pep213
SHKHDPLAYPHFLL

SEQ ID NO: 24 - protein, 14 aa, Artificial Sequence
  Synthetic - mutant pep213
SHKHDPLPAPHFLL

SEQ ID NO: 25 - protein, 14 aa, Artificial Sequence
  Synthetic - mutant pep213
SHKHDPLPYAHFLL

SEQ ID NO: 26 - protein, 14 aa, Artificial Sequence
  Synthetic - mutant pep213
SHKHDPLPYPAFLL

SEQ ID NO: 27 - protein, 14 aa, Artificial Sequence
  Synthetic - mutant pep213
SHKHDPLPYPHALL

SEQ ID NO: 28 - protein, 14 aa, Artificial Sequence
  Synthetic - mutant pep213
SHKHDPLPYPHFAL

SEQ ID NO: 29 - protein, 14 aa, Artificial Sequence
  Synthetic - mutant pep213
SHKHDPLPYPHFLA

SEQ ID NO: 30 - protein, 14 aa, Artificial Sequence
  Synthetic - mutant pep213
  MISC_FEATURE (1)..(1): X is any amino acid, preferably S or A
  MISC_FEATURE (2)..(2): X is any amino acid, preferably H or A
  MISC_FEATURE (3)..(3): X is any amino acid, preferably K or A
  MISC_FEATURE (4)..(4): X is any amino acid, preferably H or A
  MISC_FEATURE (5)..(5): X is any amino acid, preferably D or A
  MISC_FEATURE (6)..(6): X is any amino acid, preferably P or A
  MISC_FEATURE (11)..(11): X is any amino acid, preferably H or A
  MISC_FEATURE (14)..(14): X is any amino acid, preferably L or A
XXXXXXLPYPXFLX

SEQ ID NO: 31 - protein, 26 aa, Artificial Sequence
  Synthetic - pep213-linker-TAT
  MISC_FEATURE (12)..(12): X is any amino acid, preferably G or S
  REPEAT (12)..(12): X is repeated 4-8 times
YGRKKRRQRRRXSHKHDPLPYPHFLL

SEQ ID NO: 32 - protein, 26 aa, Artificial Sequence
  Synthetic - mPep213-linker (Y)-TAT
  MISC_FEATURE (12)..(12): X is any amino acid, preferably G or S
  REPEAT (12)..(12): X is repeated 4-8 times
  MISC_FEATURE (13)..(13): X is any amino acid, preferably S or A
  MISC_FEATURE (14)..(14): X is any amino acid, preferably H or A
  MISC_FEATURE (15)..(15): X is any amino acid, preferably K or A
  MISC_FEATURE (16)..(16): X is any amino acid, preferably H or A
  MISC_FEATURE (17)..(17): X is any amino acid, preferably D or A
  MISC_FEATURE (18)..(18): X is any amino acid, preferably P or A
  MISC_FEATURE (23)..(23): X is any amino acid, preferably H or A
  MISC_FEATURE (26)..(26): X is any amino acid, preferably L or A
YGRKKRRQRRRXXXXXXXLPYPXFLX

SEQ ID NO: 33 - protein, 5 aa, Artificial Sequence
  Synthetic - corePep213
  MISC_FEATURE (5)..(5): X is any amino acid, preferably A or H
LPYPX

SEQ ID NO: 34 - protein, 6 aa, Artificial Sequence
  Synthetic - Core Pep213-2
  MISC_FEATURE (5)..(5): X is any amino acid, A or H
LPYPXF

SEQ ID NO: 35 - protein, 7 aa, Artificial Sequence
  synthetic
LPYPHFL

SEQ ID NO: 36 - protein, 8 aa, Artificial Sequence
  synthetic
LPYPHFLL

SEQ ID NO: 37 - protein, 8 aa, Artificial Sequence
  synthetic
  MISC_FEATURE (1)..(1): X is from 0-30 amino acid peptide comprising any amino acids
XLPYPHFL

SEQ ID NO: 38 - protein, 7 aa, Artificial Sequence
  synthetic
  MISC_FEATURE (1)..(1): X ia 0-30 amino acid peptide comprising any amino acids
  MISC_FEATURE (7)..(7): X ia 0-30 amino acid peptide comprising any amino acids
XLPYPHX

SEQ ID NO: 39 - protein, 19 aa, Artificial Sequence
  synthetic
  MISC_FEATURE (1)..(1): X is a 0-30 amino acid peptide comprising any amino acids
  MISC_FEATURE (7)..(7): X is a 0-30 amino acid peptide comprising any amino acids
  MISC_FEATURE (8)..(8): X is a 0-10 amino acid linker.
XLPYPHXXYGRKKRRQRRR